(19) United States
(12) Patent Application Publication
(10) Pub. No.: US 2007/0065887 A1
(43) Pub. Date: Mar. 22, 2007

(54) Diagnosis of pre-cancerous conditions using pcdgf agents

(76) Inventor: Michael S. Kinch, Laytonsville, MD (US)

Correspondence Address: JONES DAY, 222 EAST 41ST ST, NEW YORK, NY, 10017, US

(21) Appl. No.: 10/565,771

(22) PCT Filed: Jul. 16, 2004
(86) PCT No.: PCT/US04/23191

§ 371 Date: Aug. 25, 2006

Related U.S. Application Data
(60) Provisional application No. 60/489,035, filed on Jul. 21, 2003.

(51) Int. Cl. G01N 33/574

(52) U.S. Cl. 435/7.23

(57) ABSTRACT

The present invention relates to methods and compositions designed for the treatment or management of pre-cancerous conditions, especially in order to prevent, delay, or decrease the likelihood that the pre-cancerous condition will progress to malignant cancer. The methods of the invention comprise the administration of an effective amount of one or more agents that decrease/inhibit PCDGF expression, secretion, and/or activity. The invention also provides pharmaceutical compositions comprising one or more PCDGF agents. In some embodiments, the PCDGF agents can be administered with other therapeutic agents for treatment or management of a pre-cancerous condition that are not PCDGF-based. Diagnostic methods and methods for screening for therapeutically useful PCDGF agents are also provided.

This application claims benefit of U.S. Provisional Application Ser. No. 60/489,035, filed Jul. 21, 2003, which is incorporated by reference herein in its entirety.

### 1. FIELD OF THE INVENTION

The present invention relates to therapeutic protocols and pharmaceutical compositions designed for the treatment or management of pre-cancerous conditions, especially in order to prevent, delay, or decrease the likelihood that the pre-cancerous condition will progress to malignant cancer. Such protocols involve the administration of an effective amount of one or more PCDGF-based therapies useful for therapy of a pre-cancerous condition. The invention also provides pharmaceutical compositions comprising one or more PCDGF agents useful for therapy of a pre-cancerous condition. Further provided by the methods of the invention are pharmaceutical compositions comprising vaccine-based therapies useful for the treatment or management of a pre-cancerous condition. Diagnostic methods and methods for screening for therapeutically useful agents of the invention are also provided.

### 2. BACKGROUND OF THE INVENTION

Cancer

A neoplasm, or tumor, is a neoplastic mass resulting from abnormal uncontrolled cell growth which can be benign or malignant. Benign tumors generally remain localized. Malignant tumors are collectively termed cancers. The term “malignant” generally means that the tumor can invade and destroy neighboring body structures and spread to distant sites to cause death (for review, see Robbins and Angell, 1976, Basic Pathology, 2d Ed., W.B. Saunders Co., Philadelphia, pp. 68-122). Cancer can arise in many sites of the body and behave differently depending upon its origin. Cancerous cells destroy the part of the body in which they originate and then spread to other part(s) of the body where they start new growth and cause more destruction.

The most life-threatening forms of cancer often arise when a population of tumor cells gains the ability to colonize distant and foreign sites in the body. These metastatic cells survive by overriding restrictions that normally constrain cell colonization into dissimilar tissues. For example, typical mammary epithelial cells will generally not grow or survive if transplanted to the lung, yet lung metastases are a major cause of breast cancer morbidity and mortality. Recent evidence suggests that dissemination of metastatic cells through the body can occur long before clinical presentation of the primary tumor. These micrometastatic cells may remain dormant for many months or years following the detection and removal of the primary tumor. Thus, a better understanding of the mechanisms that allow for the growth and survival of metastatic cells in a foreign microenvironment is critical for the improvement of therapeutics designed to fight metastatic cancer and diagnostics for the early detection and localization of metastases.

More than 1.2 million Americans develop cancer each year. Cancer is the second leading case of death in the United States and, if current trends continue, cancer is expected to be the leading cause of the death by the year 2010. Lung and prostate cancer are the top cancer killers for men in the United States. Lung and breast cancer are the top cancer killers for women in the United States. One in two men in the United States will be diagnosed with cancer at some time during his lifetime. One in three women in the United States will be diagnosed with cancer at some time during her lifetime.

A cure for cancer has yet to be found. Current treatment options, such as surgery, chemotherapy and radiation treatment, are oftentimes either ineffective or present serious side effects.

Prostate Cancer and PIN

Prostate cancer is one of the most common malignancies diagnosed in men and is the most common cancer found in men older than 60 years. A third of all men older than 50 years have a latent form of prostate cancer that may be activated into life-threatening prostate cancer. The number of men with latent prostate cancer is the same across all cultures, races, and ethnic groups, but the frequency of clinically active cancer is markedly different. Environmental factors have been implicated in activating latent prostate cancer. If cancer can be identified in the early or latent stage, the neoplastic process may be reversible.

Prostatic intraepithelial neoplasia (PIN) has been identified as a precursor lesion to prostatic carcinoma (Bostwick, 1996, Eur Urol. 30:145-52). PIN refers to the pre-cancerous end of a morphologic spectrum involving cellular proliferation within prostatic ducts, ductules, and acini. In the United States, the frequency of PIN in prostates with cancer is significantly higher than in prostates without cancer. PIN appears to predate cancer by more than 10 years, with a parallel increase in the frequency of PIN and cancer that is related to age. PIN has been found in 9% of men in the second decade of life, 22% of men in the third decade, and 40% of men in the fourth decade. By the time men reach age 80 years, the incidence of PIN is 70%. Mortality or morbidity is not directly associated with the presence of PIN. PIN does not require any specific therapy. Patients can expect to have morbidity only if they have prostate cancer. The presence of high grade PIN is important in that, upon this finding, the pathologist carefully searches the tissue specimens for evidence of cancer, and the urologist cautions the patient that continued follow-up with serum prostate specific antigen (P S A, Brawer, 1999, CA A Cancer Journal for Clinicians 49:264-81; Oesterling, 1991, Journal of Urology 145:907-23) testing and repeat biopsies is necessary. In itself, high grade PIN is not a disease that requires therapy or produces symptoms. It is a potential harbinger for the development of clinical prostatic adenocarcinoma.

PCDGF

PC Cell Derived Growth Factor (PCDGF) was first discovered as a secreted N-linked glycoprotein in the culture medium of highly tumorigenic PC cells, an insulin-independent variant isolated from the teratoma-derived adipogenic cell line 1246 (Zhou et al., 1993, J. Biol. Chem. 268, 10863-9). Determination of the amino acid sequence of PCDGF indicated similarities with the mouse granulin/epithelin precursor protein. Granulins/epithelins are 6 kDa polypeptides that belong to a family of double cysteine rich polypeptides (see e.g., Plowman et al., 1992, J. Biol. Chem. 267: 13073-8; Bateman et al., 1990, Biochem. Biophys. Res. Commun. 173, 1161-8; U.S. Pat. No. 5,416,192). Granulin/epithelin precursor polypeptide was initially thought to be processed into the small biologically active granulins/epithelins immediately after its synthesis. Additionally, the precursor polypeptide was assigned no biological activity prior to processing. However, Serrero (International Patent Publication WO 98/52607, published Nov. 26, 1998) demonstrated that the precursor polypeptide was not always processed immediately after synthesis and that it did have biological activity. Granulin/epithelin precursor polypeptide (or PCDGF) has growth promoting activity, particularly as an autocrine growth factor for the producer cells, and is implicated in tumorigenicity.

Cancer Therapy

Currently, cancer therapy may involve surgery, chemotherapy, hormonal therapy and/or radiation treatment to eradicate neoplastic cells in a patient (see, for example, Stockdale, 1998, “Principles of Cancer Patient Management”, in Scientific American: Medicine, vol. 3, Rubenstein and Federman, eds., Chapter 12, Section IV). The current standard of medical care for treating prostate cancer includes 1) radical or nerve-sparing prostatectomy in which the entire prostate gland is surgically removed, and 2) brachytherapy in which radiation seeds are implanted in the prostate gland. The cancer recurrence rate after surgery can be as high as approximately 35% at 5 years, and approximately 60% at 10 years.

Recently, cancer therapy may also involve biological therapy or immunotherapy. All of these approaches can pose significant drawbacks for the patient. Surgery, for example, may be contraindicated due to the health of the patient or may be unacceptable to the patient. Additionally, surgery may not completely remove the neoplastic tissue. Radiation therapy is only effective when the neoplastic tissue exhibits a higher sensitivity to radiation than normal tissue, and radiation therapy can also often elicit serious side effects. Hormonal therapy is rarely given as a single agent and, although it can be effective, is often used to prevent or delay recurrence of cancer after other treatments have removed the majority of the cancer cells. Biological therapies/immunotherapies are limited in number and each therapy is generally effective for a very specific type of cancer.

With respect to chemotherapy, there are a variety of chemotherapeutic agents available for treatment of cancer. A significant majority of cancer chemotherapeutics act by inhibiting DNA synthesis, either directly, or indirectly by inhibiting the biosynthesis of the deoxyribonucleotide triphosphate precursors, to prevent DNA replication and concomitant cell division (see, for example, Gilman et al., Goodman and Gilman's: The Pharmacological Basis of Therapeutics, Eighth Ed. (Pergamom Press, New York, 1990)). These agents, which include alkylating agents, such as nitrosourea, anti-metabolites, such as methotrexate and hydroxyurea, and other agents, such as etoposides, campathecins, bleomycin, doxorubicin, daunorubicin, etc., although not necessarily cell cycle specific, kill cells during S phase because of their effect on DNA replication. Other agents, specifically colchicine and the vinca alkaloids, such as vinblastine and vincristine, interfere with microtubule assembly resulting in mitotic arrest. Chemotherapy protocols generally involve administration of a combination of chemotherapeutic agents to increase the efficacy of treatment.

Despite the availability of a variety of chemotherapeutic agents, chemotherapy has many drawbacks (see, for example, Stockdale, 1998, “Principles Of Cancer Patient Management” in Scientific American Medicine, vol. 3, Rubenstein and Federman, eds., ch. 12, sect. 10). Almost all chemotherapeutic agents are toxic, and chemotherapy causes significant, and often dangerous, side effects, including severe nausea, bone marrow depression, immunosuppression, etc. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even those agents that act by mechanisms different from the mechanisms of action of the drugs used in the specific treatment; this phenomenon is termed pleiotropic drug or multidrug resistance. Thus, because of drug resistance, many cancers prove refractory to standard chemotherapeutic treatment protocols.

There is a significant need for alternative cancer treatments, particularly for treatment of cancer that has proved refractory to standard cancer treatments, such as surgery, radiation therapy, chemotherapy, and hormonal therapy. Further, it is uncommon for cancer to be treated by only one method. Thus, there is a need for development of new therapeutic agents for the treatment of cancer and new, more effective, therapy combinations for the treatment of cancer.

3. SUMMARY OF THE INVENTION

PCDGF is a secreted growth factor that is expressed in a tightly regulated manner in non-cancer cells but is overexpressed and unregulated in highly tumorigenic cells. The present invention is based, in part, on the inventor's discovery that PCDGF expression is also increased in pre-malignant conditions (e.g., prostatic intraepithelial neoplasia (PIN)). Thus, the invention encompasses therapeutic protocols for the treatment of pre-cancerous conditions comprising administration of one or more PCDGF agents. The invention also encompasses therapeutic protocols for preventing, delaying, or decreasing the likelihood that pre-cancerous conditions will progress to cancer comprising administration of one or more PCDGF agents.

In particular, PCDGF agents that i) decrease PCDGF and/or PCDGF receptor expression levels (e.g., antibodies, antisense, RNAi, etc.), ii) decrease PCDGF secretion and/or PCDGF receptor presentation (e.g., intrabodies), or iii) decrease PCDGF and/or PCDGF receptor activity (e.g., antibodies, PCDGF fragment which binds but does not activate its receptor, soluble ligand binding domain fragment of PCDGF receptor, PCDGF-based vaccines that express a PCDGF or PCDGF receptor antigenic peptide, etc.) inhibit pre-cancerous condition progression. In one embodiment, PCDGF agents are antibodies, preferably monoclonal antibodies. In a preferred embodiment, the PCDGF agent antibodies are human or humanized. In other embodiments, the invention provides methods of treating or managing a pre-cancerous condition by administering nucleic acid therapeutic agents that reduce the expression level of PCDGF or PCDGF receptor polypeptides, for example, but not by way of limitation, anti-sense nucleic acids, double stranded RNA that mediates RNA interference, ribozymes, etc. In yet other embodiments, the invention provides methods of treating or managing a pre-cancerous condition by administering PCDGF-based vaccines that express a PCDGF or PCDGF receptor antigenic peptide that reduce PCDGF and/or PCDGF receptor activity. In a preferred embodiment, the PCDGF-based vaccine is a Listeria-based vaccine.

Accordingly, the present invention relates to pharmaceutical compositions and therapeutic regimens designed to treat or manage a pre-cancerous condition associated with overexpression of PCDGF and/or PCDGF receptor or hyper-responsiveness to PCDGF in a subject comprising administering one or more PCDGF agents to the subject. In one embodiment, the pre-cancerous condition is a pre-cancerous condition of the breast, cervix, colon, esophagus, liver, lung, pancreas, prostate, skin, or stomach. In a preferred embodiment, the pre-cancerous cells overexpress PCDGF and/or PCDGF receptor or are hyper-responsive to PCDGF. In a preferred embodiment, PCDGF and/or PCDGF receptor is mislocalized in a pre-cancerous cell or in a tissue or organ (e.g., PCDGF and/or PCDGF receptor is in or expressed in areas of the body where it is not normally found because cells are inappropriately expressing PCDGF and/or PCDGF receptor). In a preferred embodiment, the methods of the invention are used to prevent, delay, or decrease the likelihood that the pre-cancerous condition progresses to cancer. In a most preferred embodiment, the pre-cancerous condition is PIN.

The PCDGF agents for use in the methods of the invention can be administered in combination with one or more therapies used to treat or manage a pre-cancerous condition that are not PCDGF-based. In particular, the present invention provides methods of treating or managing a pre-cancerous condition or preventing, delaying, or decreasing the likelihood that a pre-cancerous condition will progress to cancer in a subject comprising administering to said subject a therapeutically effective amount of one or more PCDGF agents in combination with a therapeutically effective amount of one or more chemotherapies, hormonal therapies, biological therapies/immunotherapies, radiation therapies, and/or surgery used to treat or manage a pre-cancerous condition.

The methods and compositions of the invention are useful not only in untreated patients but are also useful in the treatment of patients partially or completely refractory to current standard and experimental therapies used to treat or manage a pre-cancerous condition, including but not limited to chemotherapies, hormonal therapies, biological therapies, radiation therapies, and/or surgery as well as to improve the efficacy of such treatments. Accordingly, in a preferred embodiment, the invention provides therapeutic methods for the treatment or management of a pre-cancerous condition that has been shown to be or may be refractory or non-responsive to therapies other than those comprising administration of PCDGF agents.

The invention further provides diagnostic methods using PCDGF and/or PCDGF receptor antibodies to evaluate the efficacy of treatment or management of a pre-cancerous condition. Treatment efficacy monitored can be either therapies that are or are not based on PCDGF therapeutic agents. In general, a reduction in expression of PCDGF and/or PCDGF receptor polypeptides with a particular treatment indicates that the treatment is reducing the likelihood that the pre-cancerous condition will progress to cancer. The diagnostic methods of the invention may also be used to predict or prognose cancer. In particular embodiments, the diagnostic methods of the invention provide methods of imaging and localizing pre-cancer cancer cells and methods of diagnosis and prognosis using tissues and fluids distal to the primary tumor site (as well as methods using tissues and fluids of the primary tumor), for example, whole blood, sputum, urine, serum, fine needle aspirates (i.e., biopsies). The PCDGF antibodies and/or PCDGF receptor antibodies may also be used for immunohistochemical analyses of frozen or fixed cells or tissue assays. In another embodiment, kits comprising the pharmaceutical compositions or diagnostic reagents of the invention are provided.

3.1. Definitions

The term “agent” as used herein refers to a molecule that has a desired biological effect. Agents include, but are not limited to, proteinaceous molecules, including, but not limited to, peptides, polypeptides, proteins, post-translationally modified proteins, antibodies etc.; or a small molecule (less than 1000 daltons), an inorganic, or an organic compound; or nucleic acid molecules including, but not limited to, double-stranded or single-stranded DNA, or double-stranded or single-stranded RNA, as well as triple helix nucleic acid molecules. Agents can be derived from any known organism (including, but not limited to, animals, plants, bacteria, fungi, and protista, or viruses) or from a library of synthetic molecules.

The term “antagonist” as used herein refers to any compound that either inhibits/decreases a molecule from binding to a natural (or endogenous) binding partner or inhibits/decreases a cellular effect that results from a molecule binding to a natural (or endogenous) binding partner. In one embodiment, an antagonist inhibits/decreases a molecule (e.g., PCDGF) from binding to its natural (or endogenous) binding partner (e.g., receptor). For example, antagonists can do one or more of the following: 1) decrease/disrupt receptor-ligand binding; or 2) decrease expression such that amount of the molecule available to bind its natural (or endogenous) binding partner is decreased. In another embodiment, an antagonist inhibits/decreases a cellular effect that results from a molecule binding to its natural (or endogenous) binding partner and thus inhibits/decreases a biological effect normally observed when such binding occurs. PCDGF antagonists include, but are not limited to, biological or chemical compounds, proteins, polypeptides, peptides, antibodies, antibody fragments, nucleic acids, large or small (less than 1000 daltons) organic or inorganic molecules. PCDGF agents that antagonize PCDGF may or may not also inhibit cancer cell phenotype (e.g., colony formation in soft agar or tubular network formation in a three-dimensional basement membrane or extracellular matrix preparation).

The term “antibodies” or “antigen binding fragments thereof” as used herein refers to antibodies or antigen binding fragments thereof that specifically bind an antigen, particularly that specifically bind to a PCDGF or PCDGF receptor polypeptide or a fragment thereof and do not specifically bind to other polypeptides. Preferably, antibodies or antigen binding fragments that immunospecifically bind to a PCDGF or PCDGF receptor polypeptide or fragment thereof do not cross-react with other antigens. Antibodies or antigen binding fragments that immunospecifically bind to a PCDGF or PCDGF receptor polypeptide can be identified, for example, by immunoassays or other techniques known to those of skill in the art. Antibodies for use in the methods of the invention include, but are not limited to, synthetic antibodies, monoclonal antibodies, recombinantly produced antibodies, intrabodies, multispecific antibodies (including bi-specific antibodies), human antibodies, humanized antibodies, chimeric antibodies, single-chain Fvs (scFv) (including bi-specific scFvs), single chain antibodies Fab fragments, F(ab′) fragments, disulfide-linked Fvs (sdFv), anti-idiotypic (anti-Id) antibodies, and bispecific T cell engagers (BiTES, see Section 4.1.1, infra), and epitope-binding fragments of any of the above. In particular, antibodies for use in the methods of the present invention include immunoglobulin molecules and immunologically active portions of immunoglobulin molecules, i.e., molecules that contain an antigen binding site that immunospecifically binds to an antigen of a PCDGF or PCDGF receptor polypeptide (e.g., one or more complementarity determining regions (CDRs) of an antibody directed to a PCDGF polypeptide). Preferably, PCDGF antagonistic antibodies or antigen binding fragments thereof that immunospecifically bind to a PCDGF polypeptide or fragment thereof only antagonize PCDGF and do not significantly antagonize other activities. Preferably, PCDGF receptor antagonistic antibodies or antigen binding fragments thereof that immunospecifically bind to a PCDGF receptor polypeptide or fragment thereof only antagonize PCDGF receptor and do not significantly antagonize other activities.

The term “cancer” as used herein refers to a disease involving cells that have the potential to metastasize to distal sites and exhibit phenotypic traits that differ from those of non-cancer cells, for example, formation of colonies in a three-dimensional substrate such as soft agar or the formation of tubular networks or weblike matrices in a three-dimensional basement membrane or extracellular matrix preparation, such as MATRIGEL™. Non-cancer cells or pre-cancerous cells do not form colonies in soft agar and form distinct sphere-like structures in three-dimensional basement membrane or extracellular matrix preparations. Cancer cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless replicative potential, and sustained angiogenesis. The term “cancer cell” is meant to encompass both pre-malignant and malignant cancer cells.

The term “cancer cell phenotype inhibiting” as used herein refers to the ability of an agent to prevent or reduce cancer cell colony formation in soft agar or tubular network formation in a three-dimensional basement membrane (e.g., MATRIGEL™) or extracellular matrix preparation or any other method that detects a reduction in a cancer cell phenotype, for example, assays that detect an increase in contact inhibition of cell proliferation (e.g., reduction of colony formation in a monolayer cell culture) or reduce hyperproliferation of cancer cells. Cancer cell phenotype inhibiting compounds may also cause a reduction or elimination of colonies when added to established colonies of cancer cells in soft agar or the extent of tubular network formation in a three-dimensional basement membrane or extracellular matrix preparation. PCDGF agents may or may not have cancer cell phenotype inhibiting properties.

The term “derivative” as used herein refers to a polypeptide that comprises an amino acid sequence of a PCDGF polypeptide, a fragment of a PCDGF polypeptide, an antibody that immunospecifically binds to a PCDGF polypeptide, an antibody fragment that immunospecifically binds to a PCDGF polypeptide, PCDGF receptor polypeptide, a fragment of a PCDGF receptor polypeptide, an antibody that immunospecifically binds to a PCDGF receptor polypeptide, or an antibody fragment that immunospecifically binds to a PCDGF receptor polypeptide which has been altered by the introduction of amino acid residue substitutions, deletions or additions. The term “derivative” as used herein also refers to a polypeptide that comprises an amino acid sequence of a PCDGF polypeptide, a fragment of a PCDGF polypeptide, an antibody that immunospecifically binds to a PCDGF polypeptide, an antibody fragment that immunospecifically binds to a PCDGF polypeptide, PCDGF receptor polypeptide, a fragment of a PCDGF receptor polypeptide, an antibody that immunospecifically binds to a PCDGF receptor polypeptide, or an antibody fragment that immunospecifically binds to a PCDGF receptor polypeptide which has been modified, i.e, by the covalent attachment of any type of molecule to the polypeptide. For example, a polypeptide may be modified, e.g., by glycosylation, acetylation, pegylation, phosphorylation, amidation, derivatization by known protecting/blocking groups, proteolytic cleavage, linkage to a cellular ligand or other protein, etc. A derivative of a polypeptide may be modified by chemical modifications using techniques known to those of skill in the art, including, but not limited to specific chemical cleavage, acetylation, formylation, metabolic synthesis of tunicamycin, etc. Further, a derivative of a polypeptide may contain one or more non-classical amino acids. In one embodiment, a polypeptide derivative possesses a similar or identical function as its underivatized counterpart. In another embodiment, a derivative of polypeptide has an altered activity when compared to an underivatized counterpart. For example, a derivative antibody or fragment thereof can bind to its epitope more tightly or be more resistant to proteolysis.

The term “epitope” as used herein refers to portions of a polypeptide having antigenic or immunogenic activity in an animal, preferably in a mammal, and most preferably in a human. An epitope having immunogenic activity is a portion of a polypeptide that elicits an antibody response in an animal. An epitope having antigenic activity is a portion of a polypeptide to which an antibody immunospecifically binds as determined by any method well known in the art, for example, by immunoassays. Antigenic epitopes need not necessarily be immunogenic.

The term “fragments” as used herein includes a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of a PCDGF polypeptide, an antibody that immunospecifically binds to a PCDGF polypeptide, PCDGF receptor polypeptide, or an antibody that immunospecifically binds to a PCDGF receptor polypeptide. Preferably, PCDGF fragments are the PCDGF receptor binding domain or a portion thereof. Preferably, PCDGF receptor fragments are the extracellular domain, the PCDGF binding domain, or a portion thereof. Preferably, antibody fragments are epitope-binding fragments.

The term “humanized antibody” as used herein refers to forms of non-human (e.g., murine) antibodies that are chimeric antibodies which contain minimal sequence derived from a non-human immunoglobulin. For the most part, humanized antibodies are human immunoglobulins (recipient antibody) in which hypervariable region residues of the recipient are replaced by hypervariable region residues from a non-human species (donor antibody) such as mouse, rat, rabbit or non-human primate having the desired specificity, affinity, and capacity. In some instances, Framework Region (FR) residues of the human immunoglobulin are replaced by corresponding non-human residues. Furthermore, humanized antibodies may comprise residues which are not found in the recipient antibody or in the donor antibody. These modifications are made to further refine antibody performance. In generals the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. In some embodiments, a humanized antibody is a derivative that has been altered by the introduction of amino acid residue substitutions, deletions or additions (i.e., mutations) that immunospecifically binds to a PCDGF or PCDGF receptor polypeptide. Such a humanized antibody comprises amino acid residue substitutions, deletions or additions in one or more non-human CDRs. The humanized antibody derivative may have substantially the same binding, better binding, or worse binding when compared to a non-derivative humanized antibody. In specific embodiments, one, two, three, four, or five amino acid residues of the CDR have been substituted, deleted or added (i.e., mutated). For further details in humanizing antibodies, see European Patent Nos. EP 239,400, EP 592,106, and EP 519,596; International Publication Nos. WO 91/09967 and WO 93/17105; U.S. Pat. Nos. 5,225,539, 5,530,101, 5,565,332, 5,585,089, 5,766,886, and 6,407,213; and Padlan, 1991, Molecular Immunology 28(4/5):489-498; Studnicka et al., 1994, Protein Engineering 7(6):805-814; Roguska et al., 1994, PNAS 91:969-973; Tan et al., 2002, J. Immunol. 169:1119-25; Caldas et al., 2000, Protein Eng. 13:353-60; Morea et al., 2000, Methods 20:267-79; Baca et al., 1997, J. Biol. Chem. 272:10678-84; Roguska et al., 1996, Protein Eng. 9:895-904; Couto et al., 1995, Cancer Res. 55 (23 Supp):5973s-5977s; Couto et al., 1995, Cancer Res. 55:1717-22; Sandhu, 1994, Gene 150:409-10; Pedersen et al., 1994, J. Mol. Biol. 235:959-73; Jones et al., 1986, Nature 321:522-525; Reichmann et al., 1988, Nature 332:323-329; and Presta, 1992, Curr. Op. Struct. Biol. 2:593-596.

The term “hyper-responsive to PCDGF” as used herein refers to an increased biological response of a cell to PCDGF. In one embodiment, a cell is hyper-responsive to PCDGF due to overexpression of a PCDGF receptor. In another embodiment, a cell is hyper-responsive to PCDGF due to augmentation of PCDGF receptor signaling.

The term “hypervariable region” as used herein refers to the amino acid residues of an antibody that are responsible for antigen binding. The hypervariable region comprises amino acid residues from a “Complementarity Determining Region” or “CDR” (i.e., residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of proteins of Immunological Interest, 5th Ed. Public Health Service, National Institutes of Health, Bethesda, Md. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). “Framework Region” or “FR” residues are those variable domain residues other than the hypervariable region residues as herein defined.

The term “in combination” as used herein refers to the use of more than one therapeutic agents. The use of the term “in combination” does not restrict the order in which therapeutic agents are administered to a subject with a pre-cancerous conditions, especially PIN. A first therapeutic agent can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent to a subject which had, has, or is susceptible to a pre-cancerous condition, especially PIN. The therapeutic agents are administered to a subject in a sequence and within a time interval such that the PCDGF agent can act together with the other agent to provide an increased benefit than if they were administered otherwise. Any additional therapeutic agent can be administered in any order with the other additional therapeutic agents.

The term “inhibitor” as used herein refers to an agent that decreases or suppresses the activity of a PCDGF and/or PCDGF receptor polypeptide. Inhibitor agents can be competitive inhibitors wherein the inhibitor agent competes for binding with the endogenous (or natural) binding partner of the molecule to be inhibited. For example, competitive inhibitors can prevent receptor-ligand binding. Inhibitor agents can be non-competitive inhibitors wherein the inhibitor agent binds to the molecule to be inhibited at some site other than the endogenous binding partner site but still inhibits the action of the bound molecule. For example, non-competitive inhibitors do not prevent receptor-ligand binding but make that binding unproductive. Inhibitor agents can be un-competitive inhibitors wherein the inhibitor agent binds to and inhibits the complex of the molecule to be inhibited bound to its endogenous binding partner. For example, un-competitive inhibitors bind to the receptor-ligand complex and makes that binding unproductive.

The term “low tolerance” as used herein refers to a state in which the patient suffers from side effects from treatment so that the patient does not benefit from and/or will not continue therapy because of the adverse effects and/or the harm from the side effects outweighs the benefit of the treatment.

The terms “manage”, “managing” and “management” as used herein refer to the beneficial effects that a subject derives from a therapeutic agent, which does not result in a cure of the disease or condition. In certain embodiments, a subject is administered one or more therapeutic agents to “manage” a disease or condition so as to prevent the progression or worsening of the disease (e.g., progression of a pre-cancerous condition to cancer).

The terms “non-responsive” or “refractory” as used herein are used to describe patients treated with one or more currently available therapies (e.g., therapies used to treat or manage a pre-cancerous condition) such as chemotherapy, radiation therapy, surgery, hormonal therapy and/or biological therapy/immunotherapy, particularly a standard therapeutic regimen for the particular pre-cancerous condition, wherein the therapy is not clinically adequate to manage or treat the patients such that these patients need additional effective therapy, e.g., remain unsusceptible to therapy. The phrase can also describe patients who respond to therapy yet suffer from side effects, relapse, develop resistance, etc. The determination of whether the pre-cancer cells are “non-responsive/refractory” can be made either in vivo or in vitro by any method known in the art for assaying the effectiveness of treatment on pre-cancerous cells, using the art-accepted meanings of “refractory” in such a context.

The term “PCDGF” as used herein refers to PC cell derived growth factor. (see, e.g., International Publication No. WO 98/52607, published Nov. 26, 1998 which is incorporated herein by reference in its entirety, and Genbank Accession Nos. AY124489, NM002087, and M75161, nucleic and amino acid sequences of PCDGF are incorporated by reference in their entireties herein).

The term “PCDGF receptor” as used herein refers to receptor that can bind PC cell derived growth factor and have a biological consequence from such binding, wherein the biological consequence is one caused by PCDGF. In one embodiment, Rse is a PCDGF receptor (see, e.g., Genbank Accession Nos. BC051756, BC049368, and NM006293, nucleic and amino acid sequences of Rse are incorporated by reference in their entireties herein). See also U.S. Provisional Patent Application 60/474,493 entitled “PCDGF Receptor, Antibodies and Methods of Use” filed May 30, 2003 (Docket No. PC 200P1), U.S. Provisional Patent Application 60/478,908 entitled “PCDGF Receptor, Antibodies and Methods of Use” filed Jun. 16, 2003 (Docket No. PC 200P2), U.S. Provisional Patent Application 60/487,411 entitled “PCDGF Receptor, Antibodies and Methods of Use” filed Jul. 15, 2003 (Docket No. PC 200P3), and U.S. patent application Ser. No. 10/854,326 and PCT International Application No. PCT/US04/16547, both filed May 26, 2004, each of which is incorporated by reference in its entirety herein.

The term “PCDGF agent” as used herein is an agent of the invention that binds PCDGF, PCDGF mRNA, PCDGF receptor, or PCDGF receptor mRNA and reduces PCDGF or PCDGF receptor expression, secretion, and/or activity. In certain embodiments, the PCDGF agent of the invention is a PCDGF antagonist or inhibits/decreases binding of PCDGF to its receptor or inhibits/decreases signaling from the ligand-bound PCDGF receptor. In one embodiment, a PCDGF agent is a competitive, un-competitive, or non-competitive inhibitor of PCDGF. In another embodiment, a PCDGF agent neutralizers PCDGF such that PCDGF cannot bind its receptor. In another embodiment, a PCDGF agent binds a PCDGF receptor without causing signaling and blocks PCDGF binding. An PCDGF agent inhibits/decreases a biological effect normally observed when PCDGF binds its endogenous binding partner (e.g., receptor) such as increased cell proliferation, mitogen-activated protein (MAP) kinase activation, phosphatidylinosital 3′ kinase (PI3K) activation, focal adhesion kinase (FAK) activation, increased cyclin D1 expression, increased phosphorylation of pRB, increased expression of matrix metalloproteinase (MMP) 13 and 17. In preferred embodiments, PCDGF agents are antibodies, preferably monoclonal antibodies. In a specific embodiment, monoclonal antibodies disclosed in International Publication No. WO 98/52607, published Nov. 26, 1998, are used in the methods of the invention.

The term “potentiate” as used herein refers to an improvement in the efficacy of a therapeutic agent, e.g., by combining it with one or more other therapeutic agents. In one embodiment, combination therapies that have additive potency or an additive therapeutic effect are potentiated. In a preferred embodiment, combination therapies that have a synergistic (i.e., the effect of the combination is greater than the additive effect of the components of the combination alone) potency or synergistic therapeutic effect are potentiated. In a specific embodiment, PCDGF-based therapies are potentiated by non-PCDGF-based therapies.

The terms “pre-cancerous” or “pre-cancer” as used herein refer to cells or a condition that may (or is likely to) become cancer. Changes in cells of a pre-cancerous condition which do not signify cancer but display characteristics intermediate between non-cancer (normal) cells and cancer cells are encompassed by the term. Pre-cancerous conditions can include, but are not limited to, pre-cancerous conditions of the breast (e.g., ductal carcinoma in situ (DCIS), fibrocystic disease, fibroadenoma of the breast, lobular carcinoma in situ, intraductal hyperplasia), cervix (e.g., cervix dysplasia, squamous intraepithelial lesions (SIL)), colon (e.g., adenomatous polyps), esophagus (e.g., Barrett's esophageal dysplasia), liver (e.g., hepatocellular carcinoma, adenomatous hyperplasia), lung (e.g., atypical adenomatous hyperplasia (AAH) of the lung, lymphoma, lymphomatoid granulomatosis), pancreas (e.g., pancreatic ductal lesion, pancreatic hyperplasia, pancreatic dysplasia), prostate (e.g., prostatic intraepithelial neoplasia (PIN)), skin (e.g., xeroderma pigmentosum, carcinoma in situ of the skin, squamous cell carcinoma, solar keratosis, compound nevi, dysplastic nevi, actinic cheilitis, leukoplakia, erythroplasia, Bowen's disease, lymphomatoid papulosis), and stomach (e.g., adenomatous polyps). Pre-cancerous cells or conditions may also be known as pre-malignant or pre-invasive.

The terms “prevent”, “preventing” and “prevention” as used herein refer to the prevention of the onset, recurrence, or spread of a disease or condition in a subject resulting from the administration of a therapeutic agent.

The term “protocol” as used herein includes dosing schedules and dosing regimens.

The term “side effects” as used herein encompasses unwanted and adverse effects of a therapeutic agent. Adverse effects are always unwanted, but unwanted effects are not necessarily adverse. An adverse effect from a therapeutic agent might be harmful or uncomfortable or risky. Side effects from chemotherapy include, but are not limited to, gastrointestinal toxicity such as, but not limited to, early and late-forming diarrhea and flatulence, nausea, vomiting, anorexia, leukopenia, anemia, neutropenia, asthenia, abdominal cramping, fever, pain, loss of body weight, dehydration, alopecia, dyspnea, insomnia, dizziness, mucositis, xerostomia, and kidney failure, as well as constipation, nerve and muscle effects, temporary or permanent damage to kidneys and bladder, flu-like symptoms, fluid retention, and temporary or permanent infertility. Side effects from radiation therapy include but are not limited to fatigue, dry mouth, and loss of appetite. Side effects from biological therapies/immunotherapies include but are not limited to rashes or swellings at the site of administration, flu-like symptoms such as fever, chills and fatigue, digestive tract problems and allergic reactions. Side effects from hormonal therapies include but are not limited to nausea, fertility problems, depression, loss of appetite, eye problems, headache, and weight fluctuation. Additional undesired effects typically experienced by patients are numerous and known in the art. Many are described in the Physicians' Desk Reference (56^{th} ed., 2002).

The terms “single-chain Fv” or “scFv” as used herein refer to antibody fragments that comprise the VH and VL domains of antibody, wherein these domains are present in a single polypeptide chain. Generally, the Fv polypeptide further comprises a polypeptide linker between the VH and VL domains which enables the scFv to form the desired structure for antigen binding. For a review of sFvs see Pluckthun in The Pharmacology of Monoclonal Antibodies, vol. 113, Rosenburg and Moore eds. Springer-Verlag, New York, pp. 269-315 (1994). In specific embodiments, scFvs include bi-specific scFvs and humanized scFvs.

The terms “subject” and “patient” as used herein are used interchangeably. A subject is preferably a mammal such as a non-primate (e.g., cows, pigs, horses, cats, dogs, rats etc.) and a primate (e.g., monkey and human), most preferably a human.

The terms “treat”, “treating” and “treatment” as used herein refer to the eradication, reduction or amelioration of symptoms of a disease or condition, particularly, the eradication, removal, modification, or control of a pre-cancerous condition that results from the administration of one or more therapeutic agents. In certain embodiments, such terms refer to preventing, delaying, or decreasing the likelihood that pre-cancerous conditions will progress to cancer resulting from the administration of one or more therapeutic agents to a subject with such a disease or condition.

The term “therapeutic agent” as used herein refers to any agent that can be used in the treatment or management of a pre-cancerous condition, particularly, a pre-cancerous condition comprising cells which overexpress PCDGF and/or PCDGF receptor or are hyper-responsive to PCDGF. In certain embodiments, the term “therapeutic agent” refers to PCDGF agent. In certain other embodiments, the term “therapeutic agent” refers to non-PCDGF therapies to treat or manage a pre-cancerous condition such as chemotherapeutics, radiation therapy, hormonal therapy, biological therapy/immunotherapy.

The term “therapeutically effective amount” as used herein refers to that amount of the therapeutic agent sufficient to treat, manage, or ameliorate the symptoms of a pre-cancerous disease or condition associated with PCDGF and/or PCDGF receptor overexpression or hyper-responsiveness to PCDGF. Preferably, a therapeutic amount is the amount sufficient to destroy, modify, control, or remove pre-cancerous tissue and/or prevent, delay, or decrease the likelihood that the pre-cancerous condition will progress to cancer. A therapeutically effective amount may refer to the amount of therapeutic agent sufficient to delay or minimize the onset, recurrence or spread of the pre-cancerous condition. A therapeutically effective amount may also refer to the amount of the therapeutic agent that provides a therapeutic benefit in the treatment or management of a pre-cancerous condition. Further, a therapeutically effective amount may also refer to the amount of the therapeutic agent that provides a therapeutic benefit in preventing or decreasing the likelihood that the pre-cancerous condition will progress to cancer. Used in connection with an amount of a PCDGF agent, the term can encompass an amount that improves overall therapy, reduces or avoids unwanted effects, or enhances the therapeutic efficacy of or synergies with another (e.g., non-PCDGF-based) therapeutic agent.

### 4. DETAILED DESCRIPTION OF THE INVENTION

The present invention is based, in part, on the inventor's discovery that PCDGF is overexpressed by pre-cancerous cells, especially PIN cells. Encompassed in the methods of the invention are the administration of PCDGF agents that decrease the expression, secretion, and/or activity of PCDGF an/or PCDGF receptor to treat or manage pre-cancerous conditions, especially to prevent, delay, or decrease the likelihood that the pre-cancerous condition will progress to cancer. Also encompassed in the methods of the invention are combination therapies comprising PCDGF-based and non-PCDGF-based therapies that have additive potency or an additive therapeutic effect as well as combination therapies where the therapeutic efficacy of the combination is greater than the additive effect of the components of the combination alone. Preferably, such combinations also reduce or avoid unwanted or adverse effects.

Accordingly, the present invention relates to methods and compositions that provide for the treatment or management of pre-cancerous conditions associated with PCDGF and/or PCDGF receptor overexpression or hyper-responsiveness to PCDGF. The present invention further relates to methods and compositions that treat or manage pre-cancerous conditions of the breast (e.g., ductal carcinoma in situ (DCIS), fibrocystic disease, fibroadenoma of the breast, lobular carcinoma in situ, intraductal hyperplasia), cervix (e.g., cervix dysplasia, squamous intraepithelial lesions (SIL)), colon (e.g., adenomatous polyps), esophagus (e.g., Barrett's esophageal dysplasia), liver (e.g., hepatocellular carcinoma, adenomatous hyperplasia), lung (e.g., atypical adenomatous hyperplasia (AAH) of the lung, lymphoma, lymphomatoid granulomatosis), pancreas (e.g., pancreatic ductal lesion, pancreatic hyperplasia, pancreatic dysplasia), prostate (e.g., prostatic intraepithelial neoplasia (PIN)), skin (e.g., xeroderma pigmentosum, carcinoma in situ of the skin, squamous cell carcinoma, solar keratosis, compound nevi, dysplastic nevi, actinic cheilitis, leukoplakia, erythroplasia, Bowen's disease, lymphomatoid papulosis), and stomach (e.g., adenomatous polyps). In a specific embodiment, the pre-cancerous condition is PIN.

The present invention also relates to methods for the treatment or management of pre-cancerous conditions that has become partially or completely refractory to current treatment (e.g., a non-PCDGF-based therapy), such as chemotherapy, radiation therapy, hormonal therapy, or biological therapy.

PCDGF agents include, but are not limited to, proteinaceous molecules, including, but not limited to, peptides, polypeptides, proteins, including post-translationally modified proteins, antibodies etc.; or small molecules (less than 1000 daltons), inorganic or organic compounds; or nucleic acid molecules including, but not limited to, double-stranded or single-stranded DNA, or double-stranded or single-stranded RNA, as well as triple helix nucleic acid molecules.

4.1. Polypeptide Agents

Methods of the present invention encompass use of PCDGF agents that are polypeptides. In one embodiment, a polypeptide agent is an antibody or fragment thereof that immunospecifically binds PCDGF or PCDGF receptor polypeptides and decreases polypeptide expression, secretion, and/or activity (see International Pub. No. WO 98/52607, published Nov. 26, 1998; U.S. Pat. No. 6,309,826, issued Oct. 30, 2001; U.S. Pat. No. 6,670,183, issued Dec. 30, 2003; and U.S. Pat. No. 6,720,159, issued Apr. 13, 2004, all entitled “88 kDa Tumorigenic Growth Factor and Antagonists,” and each of which is hereby incorporated by reference in its entirety).

In another embodiment, a polypeptide agent is binding partner of PCDGF, or PCDGF receptor polypeptides such as a ligand, receptor, or fragment thereof that decreases polypeptide expression and/or function. In a specific embodiment, a polypeptide agent is a PCDGF receptor or fragment thereof (e.g., ligand binding domain which may or may not be soluble) that binds PCDGF and decreases polypeptide expression and/or function. In another specific embodiment, a polypeptide agent is a PCDGF or PCDGF fragment (e.g., receptor binding domain) that binds PCDGF receptor but does not elicit PCDGF receptor activation and/or decreases polypeptide expression and/or function.

4.1.1. Antibodies as Polypeptide Agents

The invention encompasses PCDGF agents that are antibodies (preferably monoclonal antibodies) or fragments thereof that immunospecifically bind to a PCDGF or PCDGF receptor polypeptide and decreases or inhibits polypeptide expression, secretion, and/or activity (see International Pub. No. WO 98/52607, published Nov. 26, 1998; U.S. Pat. No. 6,309,826, issued Oct. 30, 2001; U.S. Pat. No. 6,670,183, issued Dec. 30, 2003; and U.S. Pat. No. 6,720,159, issued Apr. 13, 2004, all entitled “88 kDa Tumorigenic Growth Factor and Antagonists,” and each of which is incorporated by reference herein in its entirety). Antibodies for use in methods of the invention include, but are not limited to, monoclonal antibodies, synthetic antibodies, recombinantly produced antibodies, intrabodies, multispecific antibodies (including bi-specific antibodies), human antibodies, humanized antibodies, chimeric antibodies, single-chain Fvs (scFv) (including bi-specific scFvs), single chain antibodies, Fab fragments, F(ab′) fragments, disulfide-linked Fvs (sdFv), bispecific T cell engagers, and epitope-binding fragments of any of the above. In particular, antibodies used in the methods of the present invention include immunoglobulin molecules and immunologically active portions of immunoglobulin molecules, i.e., molecules that contain an antigen binding site that immunospecifically binds to a PCDGF polypeptide and inhibits or reduces polypeptide expression, secretion, and/or activity. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The antibodies used in the methods of the invention may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice or other animals that express antibodies from human genes.

The antibodies used in the methods of the present invention may be monospecific, bispecific, trispecific or of greater multispecificity. Multispecific antibodies may immunospecifically bind to different epitopes of a PCDGF or PCDGF receptor polypeptide or may immunospecifically bind to both a PCDGF or PCDGF receptor polypeptide as well a heterologous epitope, such as a heterologous polypeptide or solid support material. See, e.g., International Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

In a preferred embodiment, antibodies for use in the methods of the invention are bispecific T cell engagers (BiTEs). Bispecific T cell engagers (BiTE) are bispecific antibodies that can redirect T cells for antigen-specific elimination of targets. A BiTE molecule has an antigen-binding domain that binds to a T cell antigen (e.g. CD3) at one end of the molecule and an antigen binding domain that will bind to an antigen on the target cell. A BiTE molecule was recently described in WO 99/54440, which is herein incorporated by reference. This publication describes a novel single-chain multifunctional polypeptide that comprises binding sites for the CD19 and CD3 antigens (CD19×CD3). This molecule was derived from two antibodies, one that binds to CD19 on the B cell and an antibody that binds to CD3 on the T cells. The variable regions of these different antibodies are linked by a polypeptide sequence, thus creating a single molecule. Also described, is the linking of the heavy chain (VH) and light chain (VL) variable domains with a flexible linker to create a single chain, bispecific antibody.

In an embodiment of this invention, an antibody or ligand that immunospecifically binds a polypeptide of interest (e.g., PCDGF or PCDGF receptor) will comprise a portion of the BiTE molecule. For example, the VH and/or VL (preferably a scFV) of an antibody that binds a polypeptide of interest (e.g., PCDGF or PCDGF receptor) can be fused to an anti-CD3 binding portion such as that of the molecule described above, thus creating a BiTE molecule that targets the polypeptide of interest (e.g., PCDGF or PCDGF receptor). In addition to the heavy and/or light chain variable domains of antibody against a polypeptide of interest (e.g., PCDGF or PCDGF receptor), other molecules that bind the polypeptide of interest (e.g., PCDGF or PCDGF receptor) can comprise the BiTE molecule, for example receptors (e.g., PCDGF receptor or PCDGF receptor). In another embodiment, the BiTE molecule can comprise a molecule that binds to other T cell antigens (other than CD3). For example, ligands and/or antibodies that immunospecifically bind to T-cell antigens like CD2, CD4, CD8, CD11a, TCR, and CD28 are contemplated to be part of this invention. This list is not meant to be exhaustive but only to illustrate that other molecules that can immunospecifically bind to a T cell antigen can be used as part of a BiTE molecule. These molecules can include the VH and/or VL portions of the antibody or natural ligands (for example LFA3 whose natural ligand is CD3). A BiTE molecule can be an antagonist.

The “binding domain” as used in accordance with the present invention denotes a domain comprising a three-dimensional structure capable of specifically binding to an epitope like native antibodies, free scFv fragments or one of their corresponding immunoglobulin chains, preferably the VH chain. Thus, said domain can comprise the VH and/or VL domain of an antibody or an immunoglobulin chain, preferably at least the VH domain or more preferably the VH and VL domain linked by a flexible polypeptide linker (scFv). On the other hand, said binding domain contained in the polypeptide of interest may comprise at least one complementarity determining region (CDR) of an antibody or immunoglobulin chain recognizing an antigen on the T cell or a cellular antigen. In this respect, it is noted that the binding domain present in the polypeptide of interest may not only be derived from antibodies but also from other T cell or cellular antigen binding protein, such as naturally occurring surface receptors or ligands. It is further contemplated that in an embodiment of the invention, said first and or second domain of the above-described polypeptide mimic or correspond to a VH and VL region from a natural antibody. The antibody providing the binding site for the polypeptide of interest can be, e.g., a monoclonal antibody, polyclonal antibody, chimeric antibody, humanized antibody, bispecific antibody, synthetic antibody, antibody fragment, such as Fab, Fv or scFv fragments etc., or a chemically modified derivative of any of these.

The antibodies used in the methods of the invention include derivatives that are modified, i.e., by the covalent attachment of any type of molecule to the antibody. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention also provides antibodies of the invention or fragments thereof that comprise a framework region known to those of skill in the art. Preferably, the antibody of the invention or fragment thereof is human or humanized.

The present invention encompasses single domain antibodies, including camelized single domain antibodies (see, e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079; which are incorporated herein by reference in their entireties). In one embodiment, the present invention provides single domain antibodies comprising two VH domains having the amino acid sequence of any of the VH domains of an antibody which immunospecifically binds a PCDGF or PCDGF receptor polypeptide and decreases polypeptide expression, expression, and/or activity with modifications such that single domain antibodies are formed. In another embodiment, the present invention also provides single domain antibodies comprising two VH domains comprising one or more of the VH CDRs of an antibody which immunospecifically binds a PCDGF or PCDGF receptor polypeptide and decreases polypeptide expression, secretion, and/or activity.

The methods of the present invention also encompass the use of antibodies or antigen binding fragments thereof that have half-lives (e.g., serum half-lives) in a mammal, preferably a human, of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present invention or fragments thereof in a mammal, preferably a human, result in a higher serum titer of said antibodies or antibody fragments in the mammal, and thus, reduce the frequency of the administration of said antibodies or antibody fragments and/or reduces the concentration of said antibodies or antibody fragments to be administered. Antibodies or antigen binding fragments thereof having increased in vivo half-lives can be generated by techniques known to those of skill in the art. For example, antibodies or antigen binding fragments thereof with increased in vivo half-lives can be generated by modifying (e.g., substituting, deleting or adding) amino acid residues identified as involved in the interaction between the Fc domain and the FcRn receptor (see, e.g., International Publication Nos. WO 97/34631 and WO 02/060919, which are incorporated by reference in their entireties herein). Antibodies or antigen binding fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

The present invention also encompasses the use of antibodies or antibody fragments comprising the amino acid sequence of one or both variable domains of an antibody which immunospecifically binds a PCDGF or PCDGF receptor polypeptide and decreases polypeptide expression, secretion, and/or activity with mutations (e.g., one or more amino acid substitutions) in the variable regions. Preferably, mutations in these antibodies maintain or enhance the avidity and/or affinity of the antibodies for the particular antigen(s) to which they immunospecifically bind. Standard techniques known to those skilled in the art (e.g., immunoassays) can be used to assay the affinity of an antibody for a particular antigen.

Standard techniques known to those skilled in the art can be used to introduce mutations in the nucleotide sequence encoding an antibody, or fragment thereof, including, e.g., site-directed mutagenesis and PCR-mediated mutagenesis, which results in amino acid substitutions. Preferably, the derivatives include less than 15 amino acid substitutions, less than 10 amino acid substitutions, less than 5 amino acid substitutions, less than 4 amino acid substitutions, less than 3 amino acid substitutions, or less than 2 amino acid substitutions relative to the original antibody or fragment thereof. In a preferred embodiment, the derivatives have conservative amino acid substitutions made at one or more predicted non-essential amino acid residues.

4.1.1.1. PCDGF Antibodies

In one embodiment, antibodies for use in the methods of the invention encompass PCDGF antibodies (preferably monoclonal antibodies) or fragments thereof that immunospecifically bind to PCDGF and decrease/inhibit PCDGF expression, secretion, and/or activity. In a specific embodiment, the antibody binds to the receptor binding domain of PCDGF and prevents PCDGF from binding to its receptor. In one embodiment, the PCDGF antibody is an antibody disclosed in International Patent Publication No. WO 98/52607. In another embodiment, the antibody immunospecifically binds an epitope in a PCDGF K19T peptide (KKVIAPRRLPDPQILKSDT; SEQ ID NO:1), S14R peptide (SARGTKCLRKKIPR; SEQ ID NO:2), or E19V peptide (EKAPAHLSLPDPQALKRDV; SEQ ID NO:3). In other embodiments, the antibody for use in the methods of the invention immunospecifically binds to PCDGF and decreases/inhibits PCDGF activity (e.g., the ability to stimulate cell proliferation, activate MAP kinase PI3K, and/or FAK, increased expression of cyclin D1 and/or MMP 13 and/or MMP 17 expression, increased phosphorylation of pRB, etc). In other embodiments, the antibody binds PCDGF with a K_{off} of less than 10⁻³ s⁻¹, less than, less than 9×10⁻⁴ s⁻¹, less than 8×10⁻⁴ s⁻¹, less than 7×10⁻⁴ s⁻¹, less than 5×10⁻⁴ s⁻¹, less than 10⁻⁴ s⁻¹, less than 9×10⁻⁵ s⁻¹, less than 5×10⁻⁵ s⁻¹, less than 10⁻⁵ s⁻¹, less than 5×10⁻⁶ s⁻¹, less than 10⁻⁶ s⁻¹, less than 5×10⁻⁷ s⁻¹, less than 10⁻⁷ s⁻¹, less than 5×10⁻⁸ s⁻¹, less than 10⁻⁸ s⁻¹, less than 5×10⁻⁹ s⁻¹, less than 10⁻⁹ s⁻¹, or less than 10⁻¹⁰ s⁻¹. In a specific embodiment, the antibody is human or has been humanized.

The present invention further encompasses the use of antibodies or antigen binding fragments thereof that immunospecifically bind to PCDGF and decrease/inhibit PCDGF expression, secretion, and/or activity, said antibodies or antibody fragments comprising one or more VH, VL, or CDRs comprising amino acid or nucleic acid residue substitutions, deletions or additions as compared to the VH, VL, or CDRs of isolated PCDGF antibodies. The antibody comprising the one or more amino acid or nucleic acid residue substitutions, deletions or additions may have substantially the same binding, better binding, or worse binding when compared to an antibody without the amino acid or nucleic acid residue substitutions, deletions or additions. In a specific embodiment, one, two, three, four, or five amino acid residues have been substituted, deleted or added (i.e., mutated). In another specific embodiments, one, two, three, four, five, six, seven, eight, nine, ten, eleven, twelve, thirteen, fourteen, or fifteen nucleic acid residues have been substituted, deleted or added (i.e., mutated). The nucleic acid substitutions may or may not change the amino acid sequence of the mutated antibody.

4.1.1.2. PCDGF Receptor Antibodies

In one embodiment, antibodies for use in the methods of the invention encompass PCDGF receptor antibodies (preferably monoclonal antibodies) or fragments thereof that immunospecifically bind to a PCDGF receptor and decrease/inhibit PCDGF receptor expression, ability to bind PCDGF, and/or activity. In a specific embodiment, the antibody binds to the ligand binding domain of a PCDGF receptor and prevents PCDGF from binding. In one embodiment, the PCDGF receptor is Rse. In other embodiments, the antibody for use in the methods of the invention immunospecifically binds to a PCDGF receptor and decreases/inhibits PCDGF activity (e.g., the ability to stimulate cell proliferation). In a most preferred embodiment, the antibody is human or has been humanized.

The present invention further encompasses the use of antibodies or antigen binding fragments thereof that immunospecifically bind to a PCDGF receptor and decrease/inhibit PCDGF receptor expression, ability to bind PCDGF, and/or activity, said antibodies or antibody fragments comprising one or more VH, VL, or CDRs comprising amino acid or nucleic acid residue substitutions, deletions or additions as compared to the VH, VL, or CDRs of isolated PCDGF receptor antibodies. The antibody comprising the one or more amino acid or nucleic acid residue substitutions, deletions or additions may have substantially the same binding, better binding, or worse binding when compared to an antibody without the amino acid or nucleic acid residue substitutions, deletions or additions. In a specific embodiment, one, two, three, four, or five amino acid residues have been substituted, deleted or added (i.e., mutated). In another specific embodiments, one, two, three, four, five, six, seven, eight, nine, ten, eleven, twelve, thirteen, fourteen, or fifteen nucleic acid residues have been substituted, deleted or added (i.e., mutated). The nucleic acid substitutions may or may not change the amino acid sequence of the mutated antibody.

4.1.1.3. Intrabodies

In certain embodiments, the antibody to be used with the invention binds to an intracellular epitope, i.e., is an intrabody. In particular, an intrabody used in the methods of the invention binds to PCDGF or PCDGF receptor and decreases/inhibits secretion of PCDGF or PCDGF receptor. An intrabody comprises at least a portion of an antibody that is capable of immunospecifically binding an antigen and preferably does not contain sequences coding for its secretion. Such antibodies will bind antigen intracellularly. In one embodiment, the intrabody comprises a single-chain Fv (“sFv”). sFvs are antibody fragments comprising the VH and VL domains of antibody, wherein these domains are present in a single polypeptide chain. Generally, the sFv polypeptide further comprises a polypeptide linker between the VH and VL domains which enables the sFv to form the desired structure for antigen binding. For a review of sFvs see Pluckthun in The Pharmacology of Monoclonal Antibodies, vol. 113, Rosenburg and Moore, eds. Springer-Verlag, New York, pp. 269-315 (1994). In a further embodiment, the intrabody preferably does not encode an operable secretory sequence and thus remains within the cell (see generally Marasco, W A, 1998, “Intrabodies: Basic Research and Clinical Gene Therapy Applications” Springer:New York).

Generation of intrabodies is well-known to the skilled artisan and is described, for example, in U.S. Pat. Nos. 6,004,940; 6,072,036; 5,965,371, which are incorporated by reference in their entireties herein. Further, the construction of intrabodies is discussed in Ohage and Steipe, 1999, J. Mol. Biol. 291:1119-1128; Ohage et al., 1999, J. Mol. Biol. 291:1129-1134; and Wirtz and Steipe, 1999, Protein Science 8:2245-2250. Recombinant molecular biological techniques such as those described for recombinant production of antibodies (e.g., Sections 4.1.1.4, 4.1.5, and 4.1.6) may also be used in the generation of intrabodies.

In one embodiment, intrabodies of the invention retain at least about 75% of the binding effectiveness of the complete antibody (i.e., having the entire constant domain as well as the variable regions) to the antigen. More preferably, the intrabody retains at least 85% of the binding effectiveness of the complete antibody. Still more preferably, the intrabody retains at least 90% of the binding effectiveness of the complete antibody. Even more preferably, the intrabody retains at least 95% of the binding effectiveness of the complete antibody.

In producing intrabodies, polynucleotides encoding the heavy and light chain variable regions of interest can be cloned by using, for example, hybridoma mRNA or splenic mRNA as a template for PCR amplification of such domains (Huse et al., 1989, Science 246:1276). In one preferred embodiment, the polynucleotides encoding the VH and VL domains are joined by a polynucleotide sequence encoding a linker to make a single chain antibody (sFv). The sFv typically comprises a single peptide with the sequence VH-linker-VL or VL-linker-VH. The linker is chosen to permit the heavy chain and light chain to bind together in their proper conformational orientation (see for example, Huston, et al., 1991, Methods in Enzym. 203:46-121). In a further embodiment, the linker can span the distance between its points of fusion to each of the variable domains (e.g., 3.5 nm) to minimize distortion of the native Fv conformation. In such an embodiment, the linker is a polypeptide of at least 5 amino acid residues, at least 10 amino acid residues, at least 15 amino acid residues, or greater. In a further embodiment, the linker should not cause a steric interference with the VH and VL domains of the combining site. In such an embodiment, the linker is 35 amino acids or less, 30 amino acids or less, or 25 amino acids or less. Thus, in a most preferred embodiment, the linker is between 15-25 amino acid residues in length. In a further embodiment, the linker is hydrophilic and sufficiently flexible such that the VH and VL domains can adopt the conformation necessary to detect antigen. Intrabodies can be generated with different linker sequences inserted between identical VH and VL domains. A linker with the appropriate properties for a particular pair of VH and VL domains can be determined empirically by assessing the degree of antigen binding for each. Examples of linkers include, but are not limited to, those sequences disclosed in Table 1.

TABLE 1

| | |
|---|---|
| Sequence | SEQ ID NO. |

| | |
|---|---|
| (Gly Gly Gly Gly Ser)₃ | SEQ ID NO:4 |
| Glu Ser Gly Arg Ser Gly Gly Gly Gly | SEQ ID NO:5 |
| Ser Gly Gly Gly Gly Ser | |
| Glu Gly Lys Ser Ser Gly Ser Gly Ser | SEQ ID NO:6 |
| Glu Ser Lys Ser Thr | |
| Glu Gly Lys Ser Ser Gly Ser Gly Ser | SEQ ID NO:7 |
| Glu Ser Lys Ser Thr Gln | |
| Glu Gly Lys Ser Ser Gly Ser Gly Ser | SEQ ID NO:8 |
| Glu Ser Lys Val Asp | |
| Gly Ser Thr Ser Gly Ser Gly Lys Ser | SEQ ID NO:9 |
| Ser Glu Gly Lys Gly | |
| Lys Glu Ser Gly Ser Val Ser Ser Glu | SEQ ID NO:10 |
| Gln Leu Ala Gln Phe Arg Ser Leu Asp | |
| Glu Ser Gly Ser Val Ser Ser Glu Glu | SEQ ID NO:11 |
| Leu Ala Phe Arg Ser Leu Asp | |

In one embodiment, intrabodies are expressed in the cytoplasm. In other embodiments, the intrabodies are localized to various intracellular locations. In such embodiments, specific localization sequences can be attached to the intrabody polypeptide to direct the intrabody to a specific location. Intrabodies can be localized, for example, to the following intracellular locations: endoplasmic reticulum (Munro et al., 1987, Cell 48:899-907; Hangejorden et al., 1991, J. Biol. Chem. 266:6015); nucleus (Lanford et al., 1986, Cell 46:575; Stanton et al., 1986, PNAS 83:1772; Harlow et al., 1985, Mol. Cell Biol. 5:1605; Pap et al., 2002, Exp. Cell Res. 265:288-93); nucleolar region (Seomi et al., 1990, J. Virology 64:1803; Kubota et al., 1989, Biochem. Biophys. Res. Comm. 162:963; Siomi et al., 1998, Cell 55:197); endosomal compartment (Bakke et al., 1990, Cell 63:707-716); mitochondrial matrix (Pugsley, A. P., 1989, “Protein Targeting”, Academic Press, Inc.); Golgi apparatus (Tang et al., 1992, J. Bio. Chem. 267:10122-6); liposomes (Letourneur et al., 1992, Cell 69:1183); peroxisome (Pap et al., 2002, Exp. Cell Res. 265:288-93); trans Golgi network (Pap et al., 2002, Exp. Cell Res. 265:288-93); and plasma membrane (Marchildon et al., 1984, PNAS 81:7679-82; Henderson et al., 1987, PNAS 89:339-43; Rhee et al., 1987, J. Virol. 61:1045-53; Schultz et al., 1984, J. Virol. 133:431-7; Ootsuyama et al., 1985, Jpn. J. Can. Res. 76:1132-5; Ratner et al., 1985, Nature 313:277-84). Examples of localization signals include, but are not limited to, those sequences disclosed in Table 2.

TABLE 2

| | | |
|---|---|---|
| Localization | Sequence | SEQ ID NO. |

| | | |
|---|---|---|
| endoplasmic | Lys Asp Glu Leu | SEQ ID NO:12 |
| reticulum | | |
| endoplasmic | Asp Asp Glu Leu | SEQ ID NO:13 |
| reticulum | | |
| endoplasmic | Asp Glu Glu Leu | SEQ ID NO:14 |
| reticulum | | |
| endoplasmic | Gln Glu Asp Leu | SEQ ID NO:15 |
| reticulum | | |
| endoplasmic | Arg Asp Glu Leu | SEQ ID NO:16 |
| reticulum | | |
| nucleus | Pro Lys Lys Lys Arg Lys | SEQ ID NO:17 |
| | Val | |
| nucleus | Pro Gln Lys Lys Ile Lys | SEQ ID NO:18 |
| | Ser | |
| nucleus | Gln Pro Lys Lys Pro | SEQ ID NO:19 |
| nucleus | Arg Lys Lys Arg | SEQ ID NO:20 |
| nucleus | Lys Lys Lys Arg Lys | SEQ ID NO:21 |
| nucleolar | Arg Lys Lys Arg Arg Gln | SEQ ID NO:22 |
| region | Arg Arg Arg Ala His Gln | |
| nucleolar | Arg Gln Ala Arg Arg Asn | SEQ ID NO:23 |
| region | Arg Arg Arg Arg Trp Arg | |
| | Glu Arg Gln Arg | |
| nucleolar | Met Pro Leu Thr Arg Arg | SEQ ID NO:24 |
| region | Arg Pro Ala Ala Ser Gln | |
| | Ala Leu Ala Pro Pro Thr | |
| | Pro | |
| endosomal | Met Asp Asp Gln Arg Asp | SEQ ID NO:25 |
| compartment | Leu Ile Ser Asn Asn Gln | |
| | Gln Leu Pro | |
| mitochondrial | Met Leu Phe Asn Leu Arg | SEQ ID NO:26 |
| matrix | Xaa Xaa Leu Asn Asn Ala | |
| | Ala Phe Arg His Gly His | |
| | Asn Phe Met Val Arg Asn | |
| | Phe Arg Cys Gly Gln Pro | |
| | Leu Xaa | |
| peroxisome | Ala Lys Leu | SEQ ID NO:27 |
| trans golgi | Ser Asp Tyr Gln Arg Leu | SEQ ID NO:28 |
| network | | |
| plasma | Gly Cys Val Cys Ser Ser | SEQ ID NO:29 |
| membrane | Asn Pro | |
| plasma | Gly Gln Thr Val Thr Thr | SEQ ID NO:30 |
| membrane | Pro Leu | |
| plasma | Gly Gln Glu Leu Ser Gln | SEQ ID NO:31 |
| membrane | His Glu | |
| plasma | Gly Asn Ser Pro Ser Tyr | SEQ ID NO:32 |
| membrane | Asn Pro | |
| plasma | Gly Val Ser Gly Ser Lys | SEQ ID NO:33 |
| membrane | Gly Gln | |
| plasma | Gly Gln Thr Ile Thr Thr | SEQ ID NO:34 |
| membrane | Pro Leu | |
| plasma | Gly Gln Thr Leu Thr Thr | SEQ ID NO:35 |
| membrane | Pro Leu | |
| plasma | Gly Gln Ile Phe Ser Arg | SEQ ID NO:36 |
| membrane | Ser Ala | |
| plasma | Gly Gln Ile His Gly Leu | SEQ ID NO:37 |
| membrane | Ser Pro | |
| plasma | Gly Ala Arg Ala Ser Val | SEQ ID NO:38 |
| membrane | Leu Ser | |
| plasma | Gly Cys Thr Len Ser Ala | SEQ ID NO:39 |
| membrane | Glu Glu | |

VH and VL domains are made up of the immunoglobulin domains that generally have a conserved structural disulfide bond. In embodiments where the intrabodies are expressed in a reducing environment (e.g., the cytoplasm), such a structural feature cannot exist. Mutations can be made to the intrabody polypeptide sequence to compensate for the decreased stability of the immunoglobulin structure resulting from the absence of disulfide bond formation. In one embodiment, the VH and/or VL domains of the intrabodies contain one or more point mutations such that their expression is stabilized in reducing environments (see Steipe et al., 1994, J: Mol. Biol. 240:188-92; Wirtz and Steipe, 1999, Protein Science 8:2245-50; Ohage and Steipe, 1999, J. Mol. Biol. 291:1119-28; Ohage et al., 1999, J. Mol Biol. 291:1129-34).

Intrabody Proteins as Therapeutics

In one embodiment, the recombinantly expressed intrabody protein is administered to a patient. Such an intrabody polypeptide must be intracellular to mediate a therapeutic effect. In this embodiment of the invention, the intrabody polypeptide is associated with a “membrane permeable sequence”. Membrane permeable sequences are polypeptides capable of penetrating through the cell membrane from outside of the cell to the interior of the cell. When linked to another polypeptide, membrane permeable sequences can also direct the translocation of that polypeptide across the cell membrane as well.

In one embodiment, the membrane permeable sequence is the hydrophobic region of a signal peptide (see, e.g., Hawiger, 1999, Curr. Opin. Chem. Biol. 3:89-94; Hawiger, 1997, Curr. Opin. Immunol. 9:189-94; U.S. Pat. Nos. 5,807,746 and 6,043,339). The sequence of a membrane permeable sequence can be based on the hydrophobic region of any signal peptide. The signal peptides can be selected, e.g., from the SIGPEP database (see e.g., von Heijne, 1987, Prot. Seq. Data Anal. 1:41-2; von Heijne and Abrahmsen, 1989, FEBS Lett. 224:439-46). When a specific cell type is to be targeted for insertion of an intrabody polypeptide, the membrane permeable sequence is preferably based on a signal peptide endogenous to that cell type. In another embodiment, the membrane permeable sequence is a viral protein (e.g., Herpes Virus Protein VP22) or fragment thereof (see e.g., Phelan et al., 1998, Nat. Biotechnol. 16:440-3). A membrane permeable sequence with the appropriate properties for a particular intrabody and/or a particular target cell type can be determined empirically by assessing the ability of each membrane permeable sequence to direct the translocation of the intrabody across the cell membrane. Examples of membrane permeable sequences include, but are not limited to, those sequences disclosed in Table 3.

TABLE 3

| | |
|---|---|
| Sequence | SEQ ID NO. |

| | |
|---|---|
| Ala Ala Val Ala Leu Leu Pro Ala Val | SEQ ID NO:40 |
| Leu Leu Ala Leu Leu Ala Pro | |
| Ala Ala Val Leu Leu Pro Val Leu Leu | SEQ ID NO:41 |
| Ala Ala Pro | |
| Val Thr Val Leu Ala Leu Gly Ala Leu | SEQ ID NO:42 |
| Ala Gly Val Gly Val Gly | |

In another embodiment, the membrane permeable sequence can be a derivative. In this embodiment, the amino acid sequence of a membrane permeable sequence has been altered by the introduction of amino acid residue substitutions, deletions, additions, and/or modifications. For example, but not by way of limitation, a polypeptide may be modified by, e.g., glycosylation, acetylation, pegylation, phosphorylation, amidation, derivatization by known protecting/blocking groups, proteolytic cleavage, linkage to a cellular ligand or other protein, etc. A derivative of a membrane permeable sequence polypeptide may be modified by chemical modifications using techniques known to those of skill in the art, including, but not limited to specific chemical cleavage, acetylation, formylation, metabolic synthesis of tunicamycin, etc. Further, a derivative of a membrane permeable sequence polypeptide may contain one or more non-classical amino acids. In one embodiment, a polypeptide derivative possesses a similar or identical function as an unaltered polypeptide. In another embodiment, a derivative of a membrane permeable sequence polypeptide has an altered activity when compared to an unaltered polypeptide. For example, a derivative membrane permeable sequence polypeptide can translocate through the cell membrane more efficiently or be more resistant to proteolysis.

The membrane permeable sequence can be attached to the intrabody in a number of ways. In one embodiment, the membrane permeable sequence and the intrabody are expressed as a fusion protein. In this embodiment, the nucleic acid encoding the membrane permeable sequence is attached to the nucleic acid encoding the intrabody using standard recombinant DNA techniques (see e.g., Rojas et al., 1998, Nat. Biotechnol. 16:370-5). In a further embodiment, there is a nucleic acid sequence encoding a spacer peptide placed in between the nucleic acids encoding the membrane permeable sequence and the intrabody. In another embodiment, the membrane permeable sequence polypeptide is attached to the intrabody polypeptide after each is separately expressed recombinantly (see e.g., Zhang et al., 1998, PNAS 95:9184-9). In this embodiment, the polypeptides can be linked by a peptide bond or a non-peptide bond (e.g. with a crosslinking reagent such as glutaraldehyde or a thiazolidino linkage see e.g., Hawiger, 1999, Curr. Opin. Chem. Biol. 3:89-94) by methods standard in the art.

The administration of the membrane permeable sequence-intrabody polypeptide can be by parenteral administration, e.g., by intravenous injection including regional perfusion through a blood vessel supplying the tissues(s) or organ(s) having the target cell(s), or by inhalation of an aerosol, subcutaneous or intramuscular injection, topical administration such as to skin wounds and lesions, direct transfection into, e.g., bone marrow cells prepared for transplantation and subsequent transplantation into the subject, and direct transfection into an organ that is subsequently transplanted into the subject. Further administration methods include oral administration, particularly when the complex is encapsulated, or rectal administration, particularly when the complex is in suppository form. A pharmaceutically acceptable carrier includes any material that is not biologically or otherwise undesirable, i.e., the material may be administered to an individual along with the selected complex without causing any undesirable biological effects or interacting in a deleterious manner with any of the other components of the pharmaceutical composition in which it is contained.

Conditions for the administration of the membrane permeable sequence-intrabody polypeptide can be readily be determined, given the teachings in the art (see e.g., Remington's Pharmaceutical Sciences, 18^{th} Ed., E. W. Martin (ed.), Mack Publishing Co., Easton, Pa. (1990)). If a particular cell type in vivo is to be targeted, for example, by regional perfusion of an organ or section of artery/blood vessel, cells from the target tissue can be biopsied and optimal dosages for import of the complex into that tissue can be determined in vitro to optimize the in vivo dosage, including concentration and time length. Alternatively, culture cells of the same cell type can also be used to optimize the dosage for the target cells in vivo.

Intrabody Gene Therapy as Therapeutic

In another embodiment, a polynucleotide encoding an intrabody is administered to a patient (e.g., as in gene therapy). In this embodiment, methods as described in Section 4.10.1 can be used to administer the intrabody polynucleotide.

4.1.1.4. Methods of Producing Antibodies

The antibodies or antigen binding fragments thereof can be produced by any method known in the art for the synthesis of antibodies, in particular, by chemical synthesis or preferably, by recombinant expression techniques.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties herein). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a PCDGF or PCDGF receptor polypeptide (either the full length polypeptide or a domain thereof, e.g., the receptor binding domain or ligand binding domain) and once an immune response is detected, e.g., antibodies specific for a PCDGF or PCDGF receptor polypeptide are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. Hybridoma clones are then assayed by methods known in the art for cells that secrete antibodies capable of binding a polypeptide of interest (e.g., PCDGF or PCDGF receptor). Ascites fluid, which generally contains high levels of antibodies, can be generated by immunizing mice with positive hybridoma clones.

Accordingly, monoclonal antibodies can be generated by culturing a hybridoma cell secreting an antibody of the invention wherein, preferably, the hybridoma is generated by fusing splenocytes isolated from a mouse immunized with a PCDGF or PCDGF receptor polypeptide or fragment thereof with myeloma cells and then screening the hybridomas resulting from the fusion for hybridoma clones that secrete an antibody able to bind a PCDGF or PCDGF receptor polypeptide.

Antibody fragments which recognize specific PCDGF or PCDGF receptor polypeptide epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to the PCDGF or PCDGF receptor epitope of interest can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177; Kettleborough et al., 1994, Eur. J. Immunol. 24:952-958; Persic et al., 1997, Gene 187:9; Burton et al., 1994, Advances in Immunology 57:191-280; International Application No. PCT/GB91/01134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

Phage may be screened for PCDGF or PCDGF receptor polypeptide binding. Ability to decrease PCDGF or PCDGF receptor expression, secretion, or activity (e.g., increased cell proliferation, MAP kinase activation, PI3K activation, FAK activation, increased cyclin D1 expression, increased phosphorylation of pRB, increased expression of MMP 13 and 17, etc.) may also be screened.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in International Publication No. WO 92/22324; Mullinax et al., 1992, BioTechniques 12:864; Sawai et al., 1995, AJRI 34:26; and Better et al., 1988, Science 240:1041 (said references incorporated by reference in their entireties herein).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g. human kappa or lambda constant regions. Preferably, the vectors for expressing the VH or VL domains comprise an EF-1α promoter, a secretion signal, a cloning site for the variable domain, constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Hunan antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of PCDGF or PCDGF receptor polypeptide. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are all incorporated by reference herein in their entireties. In addition, companies such as Abgenix, Inc. (Fremont, Calif.) and Medarex (Princeton, N.J.) can be engaged to provide human antibodies directed against a selected antigen using technology similar to that described above.

A chimeric antibody is a molecule in which different portions of the antibody are derived from different immunoglobulin molecules such as antibodies having a variable region derived from a non-human antibody and a human immunoglobulin constant region. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 6,311,415, 5,807,715, 4,816,567, and 4,816,397, which are incorporated herein by reference in their entirety. Chimeric antibodies comprising one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7:805; and Roguska et al., 1994, PNAS 91:969), and chain shuffling (U.S. Pat. No. 5,565,332).

Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; and Riechmann et al., 1988, Nature 332:323, which are incorporated by reference in their entireties herein)

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the heavy chain variable domain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG₁, IgG₂, IgG₃ and IgG₄. Usually the constant domain is a complement fixing constant domain where it is desired that the humanized antibody exhibit cytotoxic activity, and the class is typically IgG₁. Where such cytotoxic activity is not desirable, the constant domain may be of the IgG₂ class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework region (FR) and CDR sequences, more often 90%, and most preferably greater than 95%. Humanized antibodies can be produced using variety of techniques known in the art, including but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, 5,585,089, International Publication No. WO 9317105, Tan et al., 2002, J. Immunol. 169:1119-25, Caldas et al., 2000, Protein Eng. 13:353-60, Morea et al., 2000, Methods 20:267-79, Baca et al., 1997, J. Biol. Chem. 272:10678-84, Roguska et al., 1996, Protein Eng. 9:895-904, Couto et al., 1995, Cancer Res. 55 (23 Supp):5973s-5977s, Couto et al., 1995, Cancer Res. 55:1717-22, Sandhu, 1994, Gene 150:409-10, Pedersen et al., 1994, J. Mol. Biol. 235:959-73, Jones et al., 1986, Nature 321:522-525, Riechmann et al., 1988, Nature 332:323, and Presta, 1992, Curr. Op. Struct. Biol. 2:593-596. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions (see, e.g., Queen et al., U.S. Pat. No. 5,585,089; and Riechmann et al., 1988, Nature 332:323, which are incorporated by reference in their entireties herein).

Further, the antibodies of the invention can, in turn, be utilized to generate anti-idiotype antibodies using techniques well known to those skilled in the art. (See, e.g., Greenspan & Bona, 1989, FASEB J. 7:437-444; and Nissinoff, 1991, J. Immunol. 147:2429-2438). The invention provides methods employing the use of polynucleotides comprising a nucleotide sequence encoding an antibody of the invention or a fragment thereof.

4.1.2. PCDGF-Based Polypeptide Agents

In another embodiment, the polypeptide agent is a fragment of PCDGF polypeptide. Because PCDGF bound to its endogenous receptor causes an increase in cell growth or proliferation, any method that decreases the amount of PCDGF-PCDGF receptor (e.g., Rse) binding is encompassed in the methods of the invention. In one embodiment, a fragment of PCDGF which can bind to but not activate its receptor is used in the methods of the invention to inhibit binding of endogenous PCDGF to its receptor. In a specific embodiment, a fusion protein comprises a fragment of PCDGF which can bind to but not activate its receptor. In another specific embodiment, the fragment is not part of a fusion protein. Fragments of PCDGF can be made (e.g., using PCDGF sequences known in the art such as Genbank Accession Nos. AY124489, NM002087, and M75161) and assayed for the ability to bind the PCDGF receptor (e.g., Rse) or a cell expressing a PCDGF receptor. In some embodiments, the PCDGF fragment comprises the receptor binding domain. Any method known in the art to detect binging between proteins may be used including, but not limited to, affinity chromatography, size exclusion chromatography, electrophoretic mobility shift assay. Polypeptide agents of the invention that are PCDGF fragments include polypeptides that are 100%, 98%, 95%, 90%, 85%, 80%, 75%, 70%, 65%, 60%, 55%, 50%, 45%, 40% identical to endogenous PCDGF sequences. The determination of percent identity of two amino acid sequences can be determined by any method known to one skilled in the art, including BLAST protein searches.

4.1.3. PCDGF Receptor-Based Polypeptide Agents

In another embodiment, a fragment of a PCDGF receptor (e.g., Rse) which can bind PCDGF is used in the methods of the invention to inhibit binding of PCDGF to its endogenous, cell-bound receptor. In a specific embodiment, a fusion protein comprises a fragment of Rse which can bind PCDGF (e.g., the Rse fragment fused to the immunoglobulin heavy chain constant domain, see, e.g., Carles-Kinch et al., 2002, Cancer Res. 62:2840-7). In another specific embodiment, the fragment is soluble. Fragments of Rse can be made (e.g., using Rse sequences known in the art such as Genbank Accession Nos. BC051756, BC049368, and NM006293, which are incorporated by reference herein in their entireties) and assayed for the ability to bind the PCDGF. In one embodiment, the fragment comprises the extracellular domain of Rse. Any method known in the art to detect binging between proteins may be used including, but not limited to, affinity chromatography, size exclusion chromatography, electrophoretic mobility shift assay. Polypeptide agents of the invention that are Rse fragments include polypeptides that are 100%, 98%, 95%, 90%, 85%, 80%, 75%, 70%, 65%, 60%, 55%, 50%, 45%, 40% identical to endogenous Rse sequences. The determination of percent identity of two amino acid sequences can be determined by any method known to one skilled in the art, including BLAST protein searches.

4.1.4. Modified Polypeptide Agents

The polypeptide agents used in the methods of the invention (e.g., PCDGF antibodies, PCDGF receptor binding mimetics, PCDGF receptor antibodies, PCDGF ligand binding mimetics) include derivatives that are modified, Le, by the covalent attachment of any type of molecule to the polypeptide agent such that covalent attachment does not substantially alter the binding properties of the polypeptide agent. For example, but not by way of limitation, the polypeptide agent derivatives include polypeptide agents that have been modified, e.g., by glycosylation, acetylation, pegylation, phosphorylation, amidation, derivatization by known protecting/blocking groups, proteolytic cleavage, linkage to a cellular ligand or other protein, or therapeutic/detection moiety, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The methods of the present invention also encompass the use of polypeptide agents or fragments thereof that have half-lives (e.g., serum half-lives) in a mammal, preferably a human, of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the polypeptide agents in mammals, preferably humans, result in higher serum concentration of said polypeptide agents in the mammals, and thus, reduces the frequency of the administration of said polypeptide agents and/or reduces the amount of said polypeptide agents to be administered. Polypeptide agents having increased in vivo half-lives can be generated by techniques known to these of skill in the art. For example, antibody polypeptide agents with increased in vivo half-lives can be generated by modifying (e.g., substituting, deleting or adding) amino acid residues identified as involved in the interaction between the Fc domain and the FcRn receptor (see, e.g., International Patent Publication No. WO 97/34631 and U.S. patent application Ser. No. 10/020,354 filed Dec. 12, 2001 entitled “Molecules With Extended Half-Lives, Compositions and Uses Thereof,” which are incorporated by reference in their entireties herein). Polypeptide agents with increased in vivo half-lives can be generated by attaching to said polypeptide agonistic agents polymer molecules such as high molecular weight polyethyleneglycol (PEG). PEG can be attached to said polypeptide agents with or without a multifunctional linker either through site-specific conjugation of the PEG to the N- or C-terminus of said polypeptide agonistic agents or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the polypeptide agents. Unreacted PEG can be separated from polypeptide agent-PEG conjugates by, e.g., size exclusion or ion-exchange chromatography.

The methods of the present invention also encompass the use of polypeptide agents or fragments thereof that are conjugated to a therapeutic or detection moiety (see Section 4.5).

4.1.5. Polynucleotides Encoding Polypeptide Agents

Polynucleotides that encode polypeptide agents are meant to encompass polynucleotides that encode the polypeptide agents described in Sections 4.1.1, 4.1.2, 4.1.3, and 4.1.4 as well as polynucleotides that hybridize to polynucleotides which encode polypeptides agents described in Sections 4.1.1, 4.1.2, 4.1.3, and 4.1.4. Conditions for hybridization can be high stringency, intermediate stringency, or lower stringency. For example, conditions for stringent hybridization include, but are not limited to, hybridization to filter-bound DNA in 6× sodium chloride/sodium citrate (SSC) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

The polynucleotides encoding polypeptide agents for use in the methods of the invention may be obtained, and the nucleotide sequence of the polynucleotides determined, by any method known in the art. Such a polynucleotide encoding a polypeptide agent may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the polypeptide, annealing and ligating of those oligonucleotides, and then amplification of the ligated oligonucleotides by PCR. Alternatively, a polynucleotide encoding a polypeptide agent may be generated from nucleic acid from a suitable source. If a clone containing a nucleic acid encoding a particular polypeptide is not available, but the sequence of the polypeptide is known, a nucleic acid encoding the polypeptide may be chemically synthesized or obtained from a suitable source (e.g., an antibody cDNA library, or a cDNA library generated from, or nucleic acid, preferably poly A+ RNA, isolated from, any tissue or cells expressing the polypeptide of interest, such as hybridoma cells selected to express an antibody, or cells expressing a PCDGF or PCDGF receptor polypeptide) by PCR amplification using synthetic primers hybridizable to the 3′ and 5′ ends of the sequence or by cloning using an oligonucleotide probe specific for the particular sequence to identify, e.g., a cDNA clone from a cDNA library that encodes the polypeptide of interest. Amplified nucleic acids generated by PCR may then be cloned into replicable cloning vectors using any method well known in the art.

Once the nucleotide sequence of the polypeptide agent used in the methods of the invention is determined, the nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference in their entireties herein) to generate polypeptides having a different amino acid sequence, for example to create amino acid substitutions, deletions, and/or insertions.

Standard techniques known to those skilled in the art can be used to introduce mutations in the nucleotide sequence encoding a polypeptide agent, or fragment thereof, including, e.g., site-directed mutagenesis and PCR-mediated mutagenesis, which results in amino acid substitutions. Preferably, the derivatives include less than 15 amino acid substitutions, less than 10 amino acid substitutions, less than 5 amino acid substitutions, less than 4 amino acid substitutions, less than 3 amino acid substitutions, or less than 2 amino acid substitutions relative to the original polypeptide agent or fragment thereof. In a preferred embodiment, the derivatives have conservative amino acid substitutions made at one or more predicted non-essential amino acid residues. In embodiments where the polypeptide agent is an antibody or fragment thereof, the amino acid sequence may be mutated (e.g., one or more amino acid substitutions) in the framework or variable regions. Preferably, mutations in these antibodies maintain or enhance the avidity and/or affinity of the antibodies for the particular antigen to which they immunospecifically bind. Standard techniques known to those skilled in the art (e.g., immunoassays or ELISA assays) can be used to assay the degree of binding between a mutated polypeptide agent and its binding partner.

4.1.6. Recombinant Production of Polypeptide Agents

Recombinant expression of a polypeptide agent (including, but not limited to derivatives, analogs or fragments thereof) requires construction of an expression vector containing a polynucleotide that encodes the polypeptide. Once a polynucleotide encoding a polypeptide agent has been obtained, a vector for the production of the polypeptide agent may be produced by recombinant DNA technology using techniques well known in the art. Methods which are well known to those skilled in the art can be used to construct expression vectors containing polypeptide coding sequences and appropriate transcriptional and translational control signals. Thus, methods for preparing a protein by expressing a polynucleotide containing are described herein. These methods include, for example, in vitro recombinant DNA techniques, synthetic techniques, and in vivo genetic recombination. The invention, thus, provides replicable vectors comprising a nucleotide sequence encoding a PCDGF polypeptide agent operably linked to a promoter. In embodiments where the polypeptide agent is an antibody, such vectors may include the nucleotide sequence encoding the constant region of the antibody molecule (see, e.g., International Publication Nos. WO 86/05807 and WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce a polypeptide agent. Thus, the invention includes host cells containing a polynucleotide encoding a polypeptide agent or fragments thereof operably linked to a heterologous promoter.

A variety of host-expression vector systems may be utilized to express polypeptide agents (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express a polypeptide agent of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing polypeptide agonistic agent coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of a whole recombinant polypeptide agent, are used for the expression of a polypeptide agent. For example, mammalian cells such as Chinese hamster ovary cells (CHO), in conjunction with a vector such as the major intermediate early gene promoter element from human cytomegalovirus is an effective expression system for polypeptide agents, especially antibody polypeptide agents (Foecking et al., 1986, Gene 45:101; and Cockett et al., 1990, BioTechnology 8:2). In a specific embodiment, the expression of nucleotide sequences encoding a polypeptide agent is regulated by a constitutive promoter, inducible promoter or tissue specific promoter.

In bacterial systems, a number of expression vectors may be advantageously selected depending upon the use intended for the polypeptide agent being expressed. For example, when a large quantity of such a protein is to be produced, for the generation of pharmaceutical compositions, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The polypeptide agent coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the polypeptide coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the polypeptide agonistic agent in infected hosts (e.g., see Logan & Shenk, 1984, PNAS 8 1:355-359). Specific initiation signals may also be required for efficient translation of inserted polypeptide coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138, BT483, Hs578T, HTB2, BT2O, T47D, NS1, NS0, CRL7O3O HsS78Bst cells.

For long-term, high-yield production of recombinant proteins, stable expression is preferred. For example, cell lines which stably express the polypeptide agent molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the polypeptide agent. Such engineered cell lines may be particularly useful in screening and evaluation of compositions that interact directly or indirectly with the polypeptide agent.

A number of selection systems may be used, including but not limited to, the herpes simplex virus thymidine kinase (Wigler et al., 1977, Cell 11:223), glutamine synthetase, hypoxanthine guanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, gs-, hgprt- or aprt-cells, respectively. Also, antimetabolite resistance can be used as the basis of selection for the following genes: dhfr, which confers resistance to methotrexate (Wigler et al., 1980, PNAS 77:357; O'Hare et al., 1981, PNAS 78:1527); gpt, which confers resistance to mycophenolic acid (Mulligan & Berg, 1981, PNAS 78:2072); neo, which confers resistance to the aminoglycoside G-418 (Wu and Wu, 1991, Biotherapy 3:87; Tolstoshev, 1993, Ann. Rev. Pharmacol. Toxicol. 32:573; Mulligan, 1993, Science 260:926; and Morgan and Anderson, 1993, Ann. Rev. Biochem. 62: 191; May, 1993, TIB TECH 11: 155-); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference in their entireties herein.

The expression levels of a polypeptide agent can be increased by vector amplification (for a review, see Bebbington and Hentschel). The use of vectors based on gene amplification for the expression of cloned genes in mammalian cells in DNA cloning, Vol. 3. (Academic Press, New York, 1987)). When a marker in the vector system expressing polypeptide agent is amplifiable, increase in the level of inhibitor present in culture of host cell will increase the number of copies of the marker gene. Since the amplified region is associated with the nucleic acid sequence encoding the polypeptide agent, production of the polypeptide agent will also increase (Crouse et al., 1983, Mol. Cell. Biol. 3:257).

In embodiments where the polypeptide agent is an antibody, the host cell may be co-transfected with two expression vectors, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, PNAS 77:2197). The coding sequences for the heavy and light chains may comprise cDNA or genomic DNA. In some embodiments, the variable domain of a PCDGF or PCDGF receptor antibody that is a polypeptide agent or portion thereof is cloned into vectors already containing the nucleotide sequence encoding the constant region of the antibody molecule (see, e.g., U.S. Pat. Nos. 5,919,900; 5,747,296; 5,789,178; 5,591,639; 5,658,759; 5,849,522; 5,122,464; 5,770,359; 5,827,739; International Patent Publication Nos. WO 89/01036; WO 89/10404; Bebbington et al., 1992, BioTechnology 10:169).

Once a polypeptide agent has been produced by recombinant expression, it may be purified by any method known in the art for purification of a polypeptide, for example, by chromatography (e.g., ion exchange, affinity, and sizing column chromatography), centrifugation, differential solubility, or by any other standard technique for the purification of proteins. Further, the polypeptide agents may be fused to heterologous polypeptide sequences described herein or otherwise known in the art to facilitate purification.

4.2. Polynucleotide Agents

In addition polypeptide agents, nucleic acid molecules can be used in methods of the invention. Nucleic acid molecules including, but not limited to, antisense, ribozymes, and dsRNA for mediating RNA interference can be used to decrease PCDGF and/or PCDGF receptor expression. Nucleotide agents can be administered to a patient according to methods described in Section 4.10.1.

4.2.1. Antisense

The present invention encompasses antisense nucleic acid molecules (i.e., molecules which are complementary to all or part of a sense nucleic acid encoding a polypeptide of interest e.g., complementary to the coding strand of a double-stranded cDNA molecule or complementary to an mRNA sequence) for use in the methods of the present invention. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a PCDGF or PCDGF receptor polypeptide (see International Pub. No. WO 98/52607, published Nov. 26, 1998; U.S. Pat. No. 6,309,826, issued Oct. 30, 2001; U.S. Pat. No. 6,670,183, issued Dec. 30, 2003; and U.S. Pat. No. 6,720,159, issued Apr. 13, 2004, all entitled “88 kDa Tumorigenic Growth Factor and Antagonists,” and each of which is incorporated by reference herein in its entirety). The non-coding regions (“5′ and 3′ untranslated regions”) are the 5′ and 3′ sequences which flank the coding region and are not translated into amino acids.

In one embodiment, the antisense molecule is directed to PCDGF (see e.g., Genbank Accession Nos. AY124489, NM002087, and M75161). In a specific embodiment, the PCDGF antisense molecule is

| | |
|---|---|
| 5′-GGG TCC ACA TGG TCT GCC TGC-3′ | (SEQ ID NO:43) |
| or | |
| 5′-GCC ACC AGC CCT GCT GTT AAG | (SEQ ID NO:44) |
| GCC-3′. | |

In another embodiment, the antisense molecule is directed to PCDGF receptor. In a specific embodiment, the antisense molecule is directed to Rse (see e.g., Genbank Accession Nos. BC051756, BC049368, and NM006293).

An antisense oligonucleotide can be, for example, about 5, 10, 15, 20, 25, 30, 35, 40, 45 or 50 nucleotides in length. An antisense nucleic acid for use in the methods of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, i.e., PCDGF or PCDGF receptor).

The antisense nucleic acid molecules for use in the methods of the invention are typically administered to a subject or generated in situ such that they hybridize with or bind to cellular mRNA and/or genomic DNA encoding a selected PCDGF or PCDGF receptor polypeptide to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids Res. 15:6625). The antisense nucleic acid molecule can also comprise a 2′-o-methylribonucleotide (Inoue et al., 1987, Nucleic Acids Res. 15:6131) or a chimeric RNA-DNA analogue (Inoue et al., 1987, FEBS Lett. 215:327).

4.2.2. Ribozymes

The invention also encompasses the use of ribozymes in the methods of the invention. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes; described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a PCDGF or PCDGF receptor polypeptide can be designed based upon the nucleotide sequence of PCDGF or PCDGF receptor. For example, a derivative of a Tetrahymena L-19 IVS RNA can be constructed in which the nucleotide sequence of the active site is complementary to the nucleotide sequence to be cleaved in U.S. Pat. Nos. 4,987,071 and 5,116,742. Alternatively, an mRNA encoding a polypeptide of interest can be used to select a catalytic RNA having a specific ribonuclease activity from a pool of RNA molecules. See, e.g., Bartel and Szostak, 1993, Science 261:1411.

4.2.3. RNA Interference

In certain embodiments, an RNA interference (RNAi) molecule is used to decrease PCDGF or PCDGF receptor expression. RNA interference (RNAi) is the ability of double-stranded RNA (dsRNA) to suppress the expression of a gene corresponding to its own sequence (see, e.g., Cogoni and Macino, 2000, Genes Dev 10: 638-643, Guru, 2000, Nature 404, 804-808, Hammond et al., 2001, Nature Rev Gen 2: 110-119, Shi, 2003, Trends Genet. 19:9-12, U.S. Pat. No. 6,506,559, each incorporated by reference in their entireties herein). RNAi is also called post-transcriptional gene silencing or PTGS. Since the only RNA molecules normally found in the cytoplasm of a cell are molecules of single-stranded mRNA, the cell has enzymes that recognize and cut dsRNA into fragments containing 21-25 base pairs (approximately two turns of a double helix). The antisense strand of the fragment separates enough from the sense strand so that it hybridizes with the complementary sense sequence on a molecule of endogenous cellular mRNA. This hybridization triggers cutting of the mRNA in the double-stranded region, thus destroying its ability to be translated into a polypeptide. Introducing dsRNA corresponding to a particular gene thus knocks out the cell's own expression of that gene in particular tissues and/or at a chosen time.

The current models of the RNAi mechanism includes both initiation and effector steps (Hutvagner and Zamore, 2002, Curr Opin Genetics &Development 12:225-32; Hammond et al., 2001, Nature Rev Gen 2: 110-9, each incorporated by reference in their entireties herein). In the initiation step, input dsRNA is digested into 21-23 nucleotide small interfering RNAs (siRNAs), which have also been called “guide RNAs” (Sharp, 2001, Genes Dev 15: 485-490). Evidence indicates that siRNAs are produced when the enzyme Dicer, a member of the RNase III family of dsRNA-specific ribonucleases, processively cleaves dsRNA (introduced directly or via a transgene or virus) in an ATP-dependent, processive manner. Successive cleavage events degrade the RNA to 19-21 base pair duplexes (siRNAs), each with 2-nucleotide 3′ overhangs (Bernstein et al., 2001, Nature 409:363-366; Hutvagner and Zamore, 2002, Curr Opin Genetics &Development 12:225-232). In the effector step, the siRNA duplexes bind to a nuclease complex to form what is known as the RNA-induced silencing complex, or RISC. An ATP-depending unwinding of the siRNA duplex is required for activation of the RISC. The active RISC then targets the homologous transcript by base pairing interactions and cleaves the mRNA 12 nucleotides from the 3′ terminus of the siRNA. Although the mechanism of cleavage is at this date unclear, research indicates that each RISC contains a single siRNA and an RNase that appears to be distinct from Dicer (Hutvagner and Zamore, 2002, Curr Opin Genetics &Development 12:225-232). Because of the remarkable potency of RNAi in some organisms, an amplification step within the RNAi pathway has also been proposed. Amplification could occur by copying of the input dsRNAs, which would generate more siRNAs, or by replication of the siRNAs themselves. Alternatively or in addition, amplification could be effected by multiple turnover events of the RISC.

Elbashir and colleagues (Elbashir et al., 2001, Nature 411:494-8; Elbashir et al., 2001, EMBO 20:6877-88) have suggested a procedure for designing siRNAs for inducing RNAi in mammalian cells. Briefly, a 21 nucleotide sequence in the mRNA of interest that begins with an adenine-adenine (AA) dinucleotide should be identified as a potential siRNA target site. This strategy for choosing siRNA target sites is based on the observation that siRNAs with 3′ overhanging UU dinucleotides are the most effective. This is also compatible with using RNA pol III to transcribe hairpin siRNAs because RNA pol III terminates transcription at 4-6 nucleotide poly(T) tracts creating RNA molecules with a short poly(U) tail. Although siRNAs with other 3′ terminal dinucleotide overhangs have been shown to effectively induce RNAi, siRNAs with guanine residues in the overhang are not recommended because of the potential for the siRNA to be cleaved by RNase at single-stranded guanine residues. In addition to beginning with an AA dinucleotide, the siRNA target site should have a guanosine and cytidine residue percentage within the range of 30-70%. The chosen siRNA target sequence should then be subjected to a BLAST search against the EST database to ensure that only the desired gene is targeted. Various products are commercially available to aid in the preparation and use of siRNA (e.g., Ambion, Inc., Austin, Tex.).

Double-stranded (ds) RNA can be used to interfere with gene expression in mammals (Brummelkamp et al., Science 296:550-3, Krichevsky and Kosik, 2002, PNAS 99:11926-9, Paddison et al., 2002, PNAS 99:1443-8, Wianny & Zernicka-Goetz, 2000, Nature Cell Biology 2:70-75, European Patent 1144623, International Patent Publication Nos. WO 02/055693, WO 02/44321, WO 03/006,477; each incorporated by reference herein in their entireties). dsRNA is used as inhibitory RNA or RNAi of the function of PCDGF or PCDGF receptor to produce a phenotype that is the same as that of a null mutant of PCDGF or PCDGF receptor. dsRNA may also be expressed from an appropriate expression construct in the form short RNA hairpin loops to inhibit (“knock down”) expression of target mRNA sequences (e.g., PCDGF or PCDGF receptor). See, e.g., Harborth et al., 2003, Antisense Nucleic Acid Drug Dev. 13:83-105; and T7 RiboMAX™ Express RNAi System, Promega, Madison, Wis.

4.3. Small Molecule Agents

In addition polypeptide agents and nucleic acid agents, small molecules can be used in methods of the invention. Small molecules can be an organic or inorganic compound that is usually less than 1000 daltons. Small molecule agents can be derived from any known organism (including, but not limited to, animals, plants, bacteria, fungi, and protista, or viruses) or from a library of synthetic molecules. Any known method known in the art can be used to isolate PCDGF small molecule agents (see e.g., Section 4.7).

Candidate small molecule agents encompass numerous chemical classes, though typically they are organic molecules, preferably small organic compounds having a molecular weight of less than about 2,500 daltons, with molecules preferably ranging from about 100 to about 1,000 daltons being preferred. Candidate small molecule agents comprise functional groups necessary for structural interaction with proteins, particularly hydrogen bonding, and typically include at least one of an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, lipids, fatty acids, steroids, purines, pyrimidines, derivatives, structural analogs or combinations thereof.

Candidate small molecule agents are obtained from a wide variety of sources including libraries of synthetic or natural compounds. For example, numerous means are available for random and directed synthesis of a wide variety of organic compounds and biomolecules. Alternatively, libraries of natural compounds in the form of bacterial, fungal, plant and animal extracts are available or readily produced. In addition, new libraries or species of candidate agents can be made by feeding precursor molecules (e.g. chemical scaffolds) to microorganisms (including bacteria, yeast, etc.) or other organisms (plants, actinomycetes, fungi, etc.) to generate new chemicals or difficult to artificially synthesize chemicals/molecules. In a preferred embodiment, the candidate bioactive agents are organic chemical moieties, a wide variety of which are available in the literature.

In a preferred embodiment, a library of different candidate bioactive agents are used. Preferably, the library should provide a sufficiently structurally diverse population of randomized agents to effect a probabilistically sufficient range of diversity to allow binding to a particular polypeptide of interest. Accordingly, an interaction library should be large enough so that at least one of its members will have a structure that gives it affinity for the target.

Examples of methods for the synthesis of molecular libraries can be found in the art, for example in: DeWitt et al., 1993, PNAS 90:6909; Erb et al., 1994, PNAS 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233, each of which is incorporated in their entireties by reference herein.

Libraries of compounds may be presented, e.g., presented in solution (e.g., Houghten, 1992, BioTechniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, PNAS 89:1865-1869) or phage (Scott and Smith, 1990, Science 249:386-390; Devlin, 1990, Science 249:404-406; Cwirla et al., 1990, PNAS 87:6378-6382; and Felici, 1991, J. Mol. Biol. 222:301-310), each of which is incorporated by reference herein in its entirety.

As will be appreciated by those in the art, there are a wide variety of possible small molecules that can be uses in the methods of the invention. As will be appreciated by those in the art, there are a wide variety of delivery methods available, including the use of vesicles and other vehicles such as liposomes, organic solutions, dispersions, suspensions, electroporation, etc. (see, e.g., Section 4.10).

4.4. PCDGF-Based Vaccines

The present invention also relates to methods and compositions for eliciting an immune response against pre-cancerous cells, comprising the administration of an effective amount of a PCDGF agent such as a vaccine, comprising, for example, the bacterium Listeria monocytogenes (Listeria), that expresses an antigenic peptide such as PCDGF or PCDGF receptor which can be used alone or in combination with the PCDGF-based and non-PCDGF-based therapies of the present invention (see, e.g., U.S. Provisional Application Ser. No. 60/556,601, entitled “EphA2 Vaccines,” filed Mar. 26, 2004; and U.S. Provisional Application Ser. No. 60/556,631, entitled “Listeria-Based Vaccines,” filed Mar. 26, 2004, each of which is incorporated by reference herein in its entirety).

Listeria

Listeria monocytogenes (Listeria) is a Gram-positive facultative intracellular bacterium that is being developed for use in antigen-specific vaccines due to its ability to prime a potent CD4+/CD8+ T-cell mediated response via both MHC class I and class II antigen presentation pathways, and as such it has been tested recently as a vaccine vector in a human clinical trial among normal healthy volunteers.

Listeria has been studied for many years as a model for stimulating both innate and adaptive T cell-dependent antibacterial immunity. The ability of Listeria to effectively stimulate cellular immunity is based on its intracellular lifecycle. Upon infecting the host, the bacterium is rapidly taken up by phagocytes including macrophages and dendritic cells into a phagolysosomal compartment. The majority of the bacteria are subsequently degraded. Peptides resulting from proteolytic degradation of pathogens within phagosomes of infected APCs are loaded directly onto MHC class II molecules, and these MHC II-peptide complexes activate CD4+“helper” T cells that stimulate the production of antibodies, and the processed antigens are expressed on the surface of the antigen presenting cell via the class II endosomal pathway. Within the acidic compartment, certain bacterial genes are activated including the cholesterol-dependent cytolysin, LLO, which can degrade the phagolysosome, releasing the bacterium into the cytosolic compartment of the host cell, where the surviving Listeria propagate. Efficient presentation of heterologous antigens via the MHC class I pathway requires de novo endogenous protein expression by Listeria. Within antigen presenting cells (APC), proteins synthesized and secreted by Listeria are sampled and degraded by the proteosome. The resulting peptides are shuttled into the endoplasmic reticulum by TAP proteins and loaded onto MHC class I molecules. The MHC I-peptide complex is delivered to the cell surface, which in combination with sufficient co-stimulation (signal 2) activates and stimulates cytotoxic T lymphocytes (CTLs) having the cognate T cell receptor to expand and subsequently recognize the MHC I-peptide complex.

Listeria-Based Vaccines

The present invention thus relates to PCDGF agents that are PCDGF-based vaccines expressing a PCDGF or PCDGF receptor antigenic peptide that can elicit or mediate a cellular immune response, a humoral response, or both, against pre-cancerous cells that overexpress PCDGF or PCDGF receptor. In a preferred embodiment, the PCDGF-based vaccine is a Listeria-based vaccine. Where the immune response is a cellular immune response, it can be a Tc, Th1 or a Th2 immune response. In a preferred embodiment, the immune response is a Th2 cellular immune response. In another preferred embodiment, a PCDGF or PCDGF receptor antigenic peptide can be any PCDGF or PCDGF receptor antigenic peptide that is capable of eliciting an immune response against PCDGF- or PCDGF receptor-expressing cells involved in a pre-cancerous condition or a condition associated with hyperproliferating cells.

The PCDGF or PCDGF receptor antigenic peptides are preferably expressed in Listeria using a heterologous gene expression cassette. A heterologous gene expression cassette is typically comprised of the following ordered elements: (1) prokaryotic promoter; (2) Shine-Dalgarno sequence; (3) secretion signal (signal peptide); and, (4) heterologous gene. Optionally, the heterologous gene expression cassette may also contain a transcription termination sequence, in constructs for stable integration within the bacterial chromosome. While not required, inclusion of a transcription termination sequence as the final ordered element in a heterologous gene expression cassette may prevent polar effects on the regulation of expression of adjacent genes, due to read-through transcription.

The expression vectors introduced into the Listeria-based PCDGF or PCDGF receptor vaccines are preferably designed such that the Listeria-produced PCDGF or PCDGF receptor peptides and, optionally, prodrug converting enzymes, are secreted by the Listeria. A number of bacterial secretion signals are well known in the art and may be used in the compositions and methods of the present invention. Exemplary secretion signals that can be used with gram-positive microorganisms include SecA (Sadaie et al., 1991, Gene 98:101-105), SecY (Suh et al., 1990, Mol. Microbiol. 4:305-314), SecE (Jeong et al., 1993, Mol. Microbiol. 10:133-142), FtsY and FfH (PCT/NL 96/00278), and PrsA (WO 94/19471).

The promoters driving the expression of the PCDGF or PCDGF receptor antigenic peptides and, optionally, pro-drug converting enzymes, may be either constitutive, in which the peptides or enzymes are continually expressed, inducible, in which the peptides or enzymes are expressed only upon the presence of an inducer molecule(s), or cell-type specific control, in which the peptides or enzymes are expressed only in certain cell types. For example, a suitable inducible promoter can be a promoter responsible for the bacterial “SOS” response (Friedberg et al., In: DNA Repair and Mutagenesis, pp. 407-455, Am. Soc. Microbiol. Press, 1995). Such a promoter is inducible by numerous agents including chemotherapeutic alkylating agents such as mitomycin (Oda et al., 1985, Mutation Research 147:219-229; Nakamura et al., 1987, Mutation Res. 192:239-246; Shimda et al., 1994, Carcinogenesis 15:2523-2529) which is approved for use in humans. Promoter elements which belong to this group include umuC, sulA and others (Shinagawa et al., 1983, Gene 23:167-174; Schnarr et al., 1991, Biochemie 73:423-431). The sulA promoter includes the ATG of the sulA gene and the following 27 nucleotides as well as 70 nucleotides upstream of the ATG (Cole, 1983, Mol. Gen. Genet. 189:400-404). Therefore, it is useful both in expressing foreign genes and in creating gene fusions for sequences lacking initiating codons.

Although the present invention provides for the use of Listeria-based vaccines that target pre-cancerous cells that overexpress PCDGF or PCDGF receptor, also provided are PCDGF or PCDGF receptor antigenic peptide expression vehicles in the form other microorganisms.

Other microorganisms useful for the methods of the present invention that can be used as PCDGF or PCDGF receptor antigenic peptide expression vehicles, in addition to Listeria monocytogenes, include but are not limited to Borrelia burgdorferi, Brucella melitensis, Escherichia coli, enteroinvasive Escherichia coli, Legionella pneumophila, Salmonella typhi, Salmonella typhimurium, Shigella spp., Streptococcus spp., Treponema pallidum, Yersinia enterocohtica, Listeria monocytogenes, Mycobacterium avium, Mycobacterium bovis, Mycobacterium tuberculosis, BCG, Mycoplasma hominis, Rickettsiae quintana, Cryptococcus neoformans, Histoplasma capsulatum, Pneumocystis carnii, Eimeria acervulina, Neospora caninum, Plasmodium falciparum, Sarcocystis suihominis, Toxoplasma gondii, Leishmania amazonensis, Leishmania major, Leishmania mexacana, Leptomonas karyophilus, Phytomonas spp., Trypanasoma cruzi, Encephahtozoon cuniculi, Nosema helminthorum, Unikaryon legeri.

Many of the microorganisms used in the PCDGF-based vaccines of the present invention, including Listeria, are causative agents of diseases in humans and animals. For example, sepsis from gram negative bacteria is a serious problem because of the high mortality rate associated with the onset of septic shock (R. C. Bone, 1993, Clinical Microbiol. Revs. 6:57-68). Therefore, to allow the safe use of these microorganisms in both diagnostics and treatment of humans and animals, the microorganisms are attenuated in their virulence for causing disease. The end result is to reduce the risk of toxic shock or other side effects due to administration of the vector to the patient. Such attenuated microorganisms can be isolated by a number of techniques. Such methods include use of antibiotic-sensitive strains of microorganisms, mutagenesis of the microorganisms, selection for microorganism mutants that lack virulence factors, and construction of new strains of microorganisms with altered cell wall lipopolysaccharides.

In certain embodiments, the microorganisms, including Listeria, can be attenuated by the deletion or disruption of DNA sequences which encode for virulence factors which insure survival of the microorganisms in the host cell, especially macrophages and neutrophils, by, for example, homologous recombination techniques and chemical or transposon mutagenesis. Many, but not all, of these studied virulence factors are associated with survival in macrophages such that these factors are specifically expressed within macrophages due to stress, for example, acidification, or are used to induced specific host cell responses, for example, macropinocytosis, Fields et al., 1986, Proc. Natl. Acad. Sci. USA 83:5189-5193. Bacterial virulence factors include, for example: cytolysin; defensin resistance loci; DNA K; fimbriae; GroEL; inv loci; lipoprotein; LPS; lysosomal fusion inhibition; macrophage survival loci; oxidative stress response loci; pho loci (e.g., PhoP and PhoQ); pho activated genes (pag; e.g., pagB and pagC); phoP and phoQ regulated genes (prg); porins; serum resistance peptide; virulence plasmids (such as spvB, traT and ty2).

Yet another method for the attenuation of the microorganisms is to modify substituents of the microorganism which are responsible for the toxicity of that microorganism. For example, lipopolysaccharide (LPS) or endotoxin is primarily responsible for the pathological effects of bacterial sepsis. The component of LPS which results in this response is lipid A (LA). Elimination or mitigation of the toxic effects of LA results in an attenuated bacteria since 1) the risk of septic shock in the patient would be reduced and 2) higher levels of the bacterial PCDGF or PCDGF receptor antigenic peptide expression vehicle could be tolerated.

Rhodobacter (Rhodopseudomonas) sphaeroides and Rhodobacter capsulatus each possess a monophosphoryl lipid A (MLA) which does not elicit a septic shock response in experimental animals and, further, is an endotoxin antagonist. Loppnow et al., 1990, Infect. Immun. 58:3743-3750; Takayma et al., 1989, Infect. Immun. 57:1336-1338. Gram negative bacteria other than Rhodobacter can be genetically altered to produce MLA, thereby reducing its potential of inducing septic shock.

Yet another example for altering the LPS of bacteria involves the introduction of mutations in the LPS biosynthetic pathway. Several enzymatic steps in LPS biosynthesis and the genetic loci controlling them in a number of bacteria have been identified, and several mutant bacterial strains have been isolated with genetic and enzymatic lesions in the LPS pathway. In certain embodiments, the LPS pathway mutant is a firA mutant. firA is the gene that encodes the enzyme UDP-3-O(R-30 hydroxymyristoyl)-glycocyamine N-acyltransferase, which regulates the third step in endotoxin biosynthesis (Kelley et al., 1993, J. Biol. Chem. 268:19866-19874).

As a method of insuring the attenuated phenotype and to avoid reversion to the non-attenuated phenotype, the bacteria may be engineered such that it is attenuated in more than one manner, e.g., a mutation in the pathway for lipid A production and one or more mutations to auxotrophy for one or more nutrients or metabolites, such as uracil biosynthesis, purine biosynthesis, and arginine biosynthesis.

In certain embodiments of the present invention, the bacterial PCDGF or PCDGF receptor antigenic peptide expression vehicles are engineered to deliver suicide genes to the target PCDGF or PCDGF receptor-expressing cells. These suicide genes include pro-drug converting enzymes, such as Herpes simplex thymidine kinase (TK) and bacterial cytosine deaminase (CD). TK phosphorylates the non-toxic substrates acyclovir and ganciclovir, rendering them toxic via their incorporation into genomic DNA. CD converts the non-toxic 5-fluorocytosine (5-FC) into 5-fluorouracil (5-FU), which is toxic via its incorporation into RNA. Additional examples of pro-drug converting enzymes encompassed by the present invention include cytochrome p450 NADPH oxidoreductase which acts upon mitomycin C and porfiromycin (Murray et al., 1994, J. Pharmacol. Exp. Therapeut. 270:645-649). Other exemplary pro-drug converting enzymes that may be used in the methods and compositions of the present invention include: carboxypeptidase; beta-glucuronidase; penicillin-V-amidase; penicillin-G-amidase; beta-lactamase; beta.-glucosidase; nitroreductase; and carboxypeptidase A.

Where the Listeria-based vaccine comprises a microorganism that expresses a PCDGF or PCDGF receptor antigenic peptide and, optionally, a pro-drug converting enzyme, the expression constructs are preferably designed such that the microorganism-produced peptides and enzymes are secreted by the microorganism. A number of bacterial secretion signals are well known in the art and may be used in the compositions and methods of the present invention. Exemplary secretion signals that can be used with gram-positive microorganisms include SecA (Sadaie et al., Gene 98:101-105, 1991), SecY (Suh et al., Mol. Microbiol. 4:305-314, 1990), SecE (Jeong et al., Mol. Microbiol. 10:133-142, 1993), FtsY an FfH (PCT/NL 96/00278), and PrsA (WO 94/19471). Exemplary secretion signals that may be used with gram-negative microorganisms include those of soluble cytoplasmic proteins such as SecB and heat shock proteins; that of the peripheral membrane-associated protein SecA; and those of the integral membrane proteins SecY, SecE, SecD and SecF.

4.5. Targeting of Therapeutics

The present invention encompasses the use of a targeting moiety (e.g., antibody) to specifically target therapeutic agents to cells involved in the pre-cancerous disorder to be treated (e.g., pre-cancer cells). Such therapeutic agents are recombinantly fused or chemically conjugated (including both covalent and non-covalent conjugations) to a targeting moiety such as, but not limited to, antibodies or antigen binding fragments thereof. Conjugated targeting moieties can be used to target therapeutic agents to particular cell types associated with the disorder to be treated. Such targeting can improve the efficacy by increasing the concentration of targeted agent at the desired site. Also, toxicity or side effects of treatment can be minimized by reducing systemic exposure to the agent.

A conjugated agent's relative efficacy in comparison to the free agent can depend on a number of factors. For example, rate of uptake of the antibody-agent into the cell (e.g., by endocytosis), rate/efficiency of release of the agent from the antibody, rate of export of the agent from the cell, etc. can all effect the action of the agent. Antibodies used for targeted delivery of agents can be assayed for the ability to be endocytosed by the relevant cell type (i.e., the cell type associated with the disorder to be treated) by any method known in the art. Additionally, the type of linkage used to conjugate an agent to an antibody should be assayed by any method known in the art such that the agent action within the target cell is not impeded.

In some embodiments, antibodies can be fused or conjugated to liposomes, wherein the liposomes are used to encapsulate therapeutic agents (see e.g., Park et al., 1997, Can. Lett. 118:153-160; Lopes de Menezes et al., 1998, Can. Res. 58:3320-30; Tseng et al., 1999, Int. J. Can. 80:723-30; Crosasso et al., 1997, J. Pharm. Sci. 86:832-9). In a preferred embodiment, the pharmokinetics and clearance of liposomes are improved by incorporating lipid derivatives of PEG into liposome formulations (see e.g., Allen et al., 1991, Biochem Biophys Acta 1068:133-41; Huwyler et al., 1997, J. Pharmacol. Exp. Ther. 282:1541-6).

Therapeutic agents can be conjugated to antibodies by any method known in the art, including, but not limited to aldehyde/Schiff linkage, sulphydryl linkage, acid-labile linkage, cis-aconityl linkage, hydrazone linkage, enzymatically degradable linkage (see generally Garnett, 2002, Adv. Drug Deliv. Rev. 53:171-216). Additional techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58. Methods for fusing or conjugating antibodies to polypeptide agents are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, PNAS 88: 10535-10539; Zheng et al., 1995, J. Immunol. 154:5590-5600; and Vil et al., 1992, PNAS 89:11337-11341. The fusion of an antibody to an agent does not necessarily need to be direct, but may occur through linker sequences. Such linker molecules are commonly known in the art and described in Denardo et al., 1998, Clin Cancer Res. 4:2483-90; Peterson et al., 1999, Bioconjug. Chem. 10:553; Zimmerman et al., 1999, Nucl. Med. Biol. 26:943-50; Garnett, 2002, Adv. Drug Deliv. Rev. 53:171-216.

In other embodiments, antibody properties can be altered as desired (e.g., antibodies or antigen binding fragments thereof with higher affinities and lower dissociation rates) through the techniques of gene-shuffling, motif-shuffling, exon-shuffling, and/or codon-shuffling (collectively referred to as “DNA shuffling”). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16:76; Hansson, et al., 1999, J. Mol. Biol. 287:265; and Lorenzo and Blasco, 1998, BioTechniques 24:308. Antibodies or antigen binding fragments thereof, or the encoded antibodies or antigen binding fragments thereof, may be altered by being subjected to random mutagenesis by error-prone PCR, random nucleotide insertion or other methods prior to recombination. One or more portions of a polynucleotide encoding an antibody or antibody fragment, which portions immunospecifically bind to an antigen expressed on a cell associated with a particular disorder may be recombined with one or more components, motifs, sections, parts, domains, fragments, etc. of one or more heterologous molecules.

In other embodiments, the antibodies or antigen binding fragments thereof can be fused to marker sequences, such as a peptide, to facilitate purification. In preferred embodiments, the marker amino acid sequence is a hexa-histidine peptide, such as the tag provided in a pQE vector (QIAGEN, Inc., Chatsworth, Calif.), among others, many of which are commercially available (see, e.g., Gentz et al., 1989, PNAS 86:821). Other peptide tags useful for purification include, but are not limited to, the hemagglutinin (HA) tag, which corresponds to an epitope derived from the influenza hemagglutinin protein (Wilson et al., 1984, Cell 37:767) and the “flag” tag. Any purification method known in the art can be used (see e.g., International Publication WO 93/21232; EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452).

In one embodiment, the antibody used to target the therapeutic is a PCDGF antibody. In another embodiment, the moiety used to target the therapeutic is a receptor binding fragment of PCDGF. In another embodiment, the moiety used to target the therapeutic is a PCDGF receptor antibody. Pre-cancerous cells overexpress PCDGF polypeptides and/or are hyper-responsive (e.g., overexpress PCDGF receptors) to PCDGF thus are good candidates to use to target therapeutic agents to pre-cancerous cells rather than non-cancer cells. An antibody or antigen binding fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, a PCDGF or PCDGF receptor antibody or antigen binding fragment thereof or a receptor binding fragment of PCDGF may be conjugated to a therapeutic agent or drug moiety that modifies a given biological response. Therapeutic agents or drug moieties are not to be construed as limited to classical chemical therapeutic agents. For example, the drug moiety may be a molecule (e.g., protein, polypeptide, nucleic acid, etc.) possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)). In other embodiments, the moiety possessing a desired biological activity is a PCDGF agent.

Moreover, an antibody can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50 each incorporated by reference in their entireties herein.

Alternatively, an antibody can be conjugated to a second antibody to form an antibody heteroconjugate as described by Segal in U.S. Pat. No. 4,676,980.

In other embodiments, antibodies of the invention or fragments or variants thereof can be conjugated to a diagnostic or detectable agent. Such antibodies can be useful for monitoring or prognosing the development or progression of a pre-cancerous condition as part of a clinical testing procedure, such as determining the efficacy of a particular therapy. Such diagnosis and detection can accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹ Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

In another embodiment, PCDGF agents can be conjugated to an antibody that does not immunospecifically bind a PCDGF polypeptide but targets pre-cancerous cells by immunospecifically binding to an epitope only expressed or overexpressed on pre-cancerous cells (e.g., EphA2). Examples of such monoclonal antibodies that immunospecifically bind tumor-associated antigens expressed at a higher density on pre-cancerous cells relative to non-cancer cells can be found in the art.

4.6. Therapeutic Methods

The present invention encompasses methods for treating or managing pre-cancerous conditions, especially in order to prevent, delay, or decrease the likelihood that the pre-cancerous condition will progress to malignant cancer, in a subject comprising administering one or more PCDGF agents. In some embodiments, one or more PCDGF agents are administered with one or more non-PCDGF-based therapeutics. In a specific embodiment, the disorder to be treated or managed is a pre-cancerous condition associated with cells that overexpress PCDGF and/or PCDGF receptor polypeptide. In a another specific embodiment, the disorder to be treated or managed is a pre-cancerous condition associated with pre-cancer cells that are hyper-responsive to PCDGF. In more specific embodiments, the pre-cancerous condition is a pre-cancerous condition of the breast (e.g., ductal carcinoma in situ (DCIS), fibrocystic disease, fibroadenoma of the breast, lobular carcinoma in situ, intraductal hyperplasia), cervix (e.g., cervix dysplasia, squamous intraepithelial lesions (SIL)), colon (e.g., adenomatous polyps), esophagus (e.g., Barrett's esophageal dysplasia), liver (e.g., hepatocellular carcinoma, adenomatous hyperplasia), lung (e.g., atypical adenomatous hyperplasia (AAH) of the lung, lymphoma, lymphomatoid granulomatosis), pancreas (e.g., pancreatic ductal lesion, pancreatic hyperplasia, pancreatic dysplasia), prostate (e.g., prostatic intraepithelial neoplasia (PIN)), skin (e.g., xeroderma pigmentosum, carcinoma in situ of the skin, squamous cell carcinoma, solar keratosis, compound nevi, dysplastic nevi, actinic cheilitis, leukoplakia, erythroplasia, Bowen's disease, lymphomatoid papulosis), or stomach (e.g., adenomatous polyps).

In some embodiments, the one or more PCDGF agents for use in the methods of the invention are antibodies. In preferred embodiments, the PCDGF agent antibodies for use in the methods of the invention are human or have been humanized. In other embodiments, variants of PCDGF agent antibodies e.g., with one or more amino acid substitutions, particularly in the variable domain, that have increased activity, binding ability, etc., as compared to non-variant PCDGF agent antibodies are used in the methods of the invention.

In another specific embodiment, the therapeutic methods of the invention comprise administration of a PCDGF agent that inhibits expression of PCDGF or PCDGF receptor. Such agents include but are not limited to, antisense nucleic acids specific PCDGF, double stranded PCDGF RNA that mediates RNAi, anti-PCDGF ribozymes, antisense nucleic acids specific PCDGF receptor, double stranded PCDGF receptor RNA that mediates RNAi, anti-PCDGF receptor ribozymes, etc. (see Section 4.2) or small molecule inhibitors of PCDGF and/or PCDGF receptor activity.

In some embodiments, the PCDGF agents for use in the methods of the invention are administered concurrently with one or more non-PCDGF-based therapeutics used to treat pre-cancerous conditions. The term “concurrently” is not limited to the administration of PCDGF agents and non-PCDGF-based therapeutic agents at exactly the same time, but rather it is meant that the PCDGF agents and the non-PCDGF-based therapeutic agents are administered to a subject in a sequence and within a time interval such that the agents can act together with one another to provide an increased benefit than if they were administered otherwise. For example, each therapeutic agent may be administered at the same time or sequentially in any order at different points in time; however, if not administered at the same time, they should be administered sufficiently close in time so as to provide the desired therapeutic effect. Each therapeutic agent can be administered separately, in any appropriate form and by any suitable route. In some embodiments, the PCDGF agents for use in the methods of the invention can be administered before, concurrently or after surgery. Preferably the surgery completely removes localized pre-cancerous cells. Surgery can also be done as a preventive measure or to relieve pain.

In various embodiments, the therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more components are administered within the same patient visit.

The dosage amounts and frequencies of administration provided herein are encompassed by the terms therapeutically effective. The dosage and frequency further will typically vary according to factors specific for each patient depending on the specific therapeutic agents administered, the severity and type of pre-cancerous condition, the route of administration, as well as age, body weight, response, and the past medical history of the patient. Suitable regimens can be selected by one skilled in the art by considering such factors and by following, for example, dosages reported in the literature and recommended in the Physician's Desk Reference (56^{th} ed., 2002).

4.6.1. Patient Population

The present invention encompasses methods for treating or managing pre-cancerous conditions, especially in order to prevent, delay, or decrease the likelihood that the pre-cancerous condition will progress to malignant cancer, in a subject comprising administering one or more PCDGF agents. The subject is preferably a mammal such as non-primate (e.g., cows, pigs, horses, cats, dogs, rats, etc.) and a primate (e.g., monkey, such as a cynomolgous monkey and a human). In a preferred embodiment, the subject is a human.

In one embodiment, the methods of the invention comprise the administration of one or more PCDGF agents to patients suffering from a pre-cancerous condition. Examples of patients having specific pre-cancerous conditions that can be treated by the methods encompassed by the invention include, but are not limited to, pre-cancerous conditions in which pre-cancer cells overexpress a PCDGF and/or PCDGF receptor polypeptide and/or are hyper-responsive to PCDGF. Examples of such pre-cancerous conditions are pre-cancerous conditions of the breast, cervix, colon, esophagus, liver, lung, pancreas, prostate, skin, or stomach. Specific examples of pre-cancerous conditions include, but are not limited to, prostatic intraepithelial neoplasia (PIN), ductal carcinoma in situ (DCIS), fibrocystic disease, fibroadenoma of the breast, lobular carcinoma in situ, intraductal hyperplasia, cervix dysplasia, squamous intraepithelial lesions (SIL), adenomatous polyps, Barrett's esophageal dysplasia, hepatocellular carcinoma, adenomatous hyperplasia, atypical adenomatous hyperplasia (AAH) of the lung, lymphoma, lymphomatoid granulomatosis, pancreatic ductal lesion, pancreatic hyperplasia, pancreatic dysplasia, xeroderma pigmentosum, carcinoma in situ of the skin, squamous cell carcinoma, solar keratosis, compound nevi, dysplastic nevi, actinic cheilitis, leukoplakia, erythroplasia, Bowen's disease, and lymphomatoid papulosis. In particular embodiments, methods of the invention can be used to treat patients having a pre-cancerous condition to prevent, delay, or decrease the likelihood that the pre-cancerous condition will progress to malignant cancer.

The methods and compositions of the invention may be used as a first line or second line of treatment. Included in the invention are also methods for the treatment of patients undergoing non-PCDGF-based therapies for pre-cancerous conditions. The methods of the invention can be used before any adverse effects or intolerance of these other therapies for pre-cancerous conditions occurs. Non-PCDGF-based therapies for pre-cancerous conditions include, but are not limited to, chemotherapy, radiation therapy, hormonal therapy, biological therapy/immunotherapy, surgery (see e.g., Section 4.6.2).

The invention also encompasses methods for administering one or more PCDGF agents to treat, manage, or ameliorate symptoms in patients refractory to one or more therapies that are not PCDGF-based for pre-cancerous conditions. In a certain embodiment, that a pre-cancerous condition is refractory to a therapy means that at least some significant portion of the pre-cancer cells are not prevented from progressing to displaying characteristics of a cancer cell. The determination of whether the pre-cancer cells are refractory can be made either in vivo or in vitro by any method known in the art for assaying the effectiveness of treatment on pre-cancer cells, using the art-accepted meanings of “refractory” in such a context. In various embodiments, a pre-cancer cell is refractory where the number of pre-cancer cells has not been significantly reduced, or has increased. Among these patients are refractory patients and those with pre-cancer despite treatment with existing therapies for pre-cancerous conditions.

In another embodiment, the methods and compositions of the invention comprise the administration of one or more PCDGF agents to patients expected to suffer from a pre-cancerous condition, e.g., have a genetic predisposition for a particular type of pre-cancerous condition or cancer. Such patients may or may not have been previously treated for a pre-cancerous condition. In other embodiments, the patients have been treated previously for a pre-cancerous condition and currently have no disease activity. In other embodiments, one or more PCDGF agents are administered to prevent the recurrence of a pre-cancerous condition.

In other embodiments, the invention also provides methods of treatment of pre-cancerous conditions as alternatives to current (non-PCDGF-based) therapies. In one embodiment, the current therapy has proven or may prove too toxic (i.e., results in unacceptable or unbearable side effects) for the patient. In another embodiment, the PCDGF-based therapy has decreased side effects as compared to the current therapy. In another embodiment, the patient has proven refractory to the current therapy. In such embodiments, the invention provides administration of one or more PCDGF agents of the invention without any other non-PCDGF-based therapy for pre-cancerous conditions. In certain embodiments, one or more PCDGF agents of the invention can be administered to a patient in need thereof instead of another therapy to treat pre-cancerous condition.

4.6.2. Other Therapeutic Agents

In some embodiments, the invention encompasses methods for administering PCDGF agents in combination with non-PCDGF-based therapies for pre-cancerous conditions (such as, but not limited to, chemotherapies, radiation therapies, hormonal therapies, and/or biological therapies/immunotherapies) to treat, manage, or ameliorate pre-cancerous conditions in patients. In some specific embodiments, dosages of non-PCDGF-based therapies for pre-cancerous conditions can be reduced due to combination therapy with PCDGF agents, e.g., to decrease adverse effects or intolerance of these other non-PCDGF-based therapies.

In one embodiment, the pre-cancerous condition is high grade prostatic intraepithelial neoplasia (PIN) and the non-PCDGF based therapy is, e.g., raloxifene, radiation therapy, or interstitial implantation of radioisotopes (e.g., I-125, palladium, iridium).

In another embodiment, the pre-cancerous condition is ductal carcinoma in situ (DCIS) and the non-PCDGF based therapy is, e.g., tamoxifen, aromatase inhibitors (e.g., anastrozole), or surgical removal.

In another embodiment, the pre-cancerous condition is fibrocystic disease and the non-PCDGF based therapy is, e.g., progesterone cream, iodine, aromatase inhibitors (e.g., anastrozole), or Vitamins E and B6.

In another embodiment, the pre-cancerous condition is fibroadenoma of the breast and the non-PCDGF-based therapy is, e.g., tamoxifen, aromatase inhibitors (e.g., anastrozole), flax oil, fish oil, Vitamins E and C, danazol, Magnetic Resonance Guided Focused Ultrasound Therapy or surgical removal.

In another embodiment, the pre-cancerous condition is lobular carcinoma in situ and the non-PCDGF based therapy is, e.g., tamoxifen, aromatase inhibitors (e.g., anastrozole), raloxifene, or surgical removal.

In another embodiment, the pre-cancerous condition is intraductal hyperplasia and the non-PCDGF based therapy is, e.g., tamoxifen or aromatase inhibitors (e.g., anastrozole).

In another embodiment, the pre-cancerous condition is cervix dysplasia and the non-PCDGF based therapy is, e.g., folic acid, Vitamin A, beta-carotene, electrocauterization, cryosurgery, laser vaporization, or surgical removal.

In another embodiment, the pre-cancerous condition is squamous intraepithelial lesions (SIL) and the non-PCDGF based therapy is, e.g., cryotherapy or laser ablation.

In another embodiment, the pre-cancerous condition is adenomatous polyps and the non-PCDGF based therapy is, e.g., Celecoxib or surgical removal.

In another embodiment, the pre-cancerous condition is Barrett's esophageal dysplasia and the non-PCDGF based therapy is, e.g., Celecoxib, Prilosec, balloon photodynamic therapy after Photofrin® treatment, or surgical removal.

In another embodiment, the pre-cancerous condition is hepatocellular carcinoma and the non-PCDGF based therapy is, e.g., percutaneous ethanol injection, antineoplastic agents mixed with iodized oil (e.g., Lipiodol®), tetrathiomolybdate, or adoptive immunotherapy with interleukin-2 and anti-CD3 activated autologous lymphocytes.

In another embodiment, the pre-cancerous condition is adenomatous hyperplasia and the non-PCDGF based therapy is, e.g., cyclic medroxyprogesterone acetate.

In another embodiment, the pre-cancerous condition is lymphoma and the non-PCDGF based therapy is, e.g., Rituxan, Zevalin, Bexxar, Oncolym, or radiation therapy.

In another embodiment, the pre-cancerous condition is lymphomatoid granulomatosis and the non-PCDGF based therapy is, e.g., interferon (IFN) alpha-2b.

In another embodiment, the pre-cancerous condition is pancreatic ductal lesion and the non-PCDGF based therapy is, e.g., 5-fluorouracil or irradiation.

In another embodiment, the pre-cancerous condition is pancreatic hyperplasia and the non-PCDGF based therapy is, e.g., Proscar.

In another embodiment, the pre-cancerous condition is pancreatic dysplasia and the non-PCDGF based therapy is, e.g., Proscar.

In another embodiment, the pre-cancerous condition is xeroderma pigmentosum and the non-PCDGF based therapy is, e.g., oral retinoids, 5-fluorouracil, or topical formulation of a bacterial T4 endonuclease.

In another embodiment, the pre-cancerous condition is carcinoma in situ of the skin and the non-PCDGF based therapy is, e.g., systemic retinoids, 5-fluorouracil cream, interferon-α.

In another embodiment, the pre-cancerous condition is squamous cell carcinoma and the non-PCDGF based therapy is, e.g., topical immune stimulant (such as 5% imiquimod cream), systemic retinoids, 5-fluorouracil cream, interferon-α, or surgical removal.

In another embodiment, the pre-cancerous condition is solar keratosis and the non-PCDGF based therapy is, e.g., antioxidants such as Vitamins A (e.g., Retin-A® or retinol), C, E and beta-carotene, exfoliating agents (such as hydroxy acids), aminolevulinic acid (e.g., Levulan Kerastick), 5-fluorouracil cream, systemic retinoids, interferon-α, or freezing with liquid nitrogen.

In another embodiment, the pre-cancerous condition is compound nevi and the non-PCDGF based therapy is, e.g., systemic retinoids, 5-fluorouracil cream, interferon-α, or excision.

In another embodiment, the pre-cancerous condition is dysplastic nevi and the non-PCDGF based therapy is, e.g., systemic retinoids, 5-fluorouracil cream, interferon-α.

In another embodiment, the pre-cancerous condition is actinic cheilitis and the non-PCDGF based therapy is, e.g., 5-aminolevulinic acid, topical immune stimulant (such as 5% imiquimod cream), systemic retinoids, 5-fluorouracil cream, interferon-α, or photodynamic therapy.

In another embodiment, the pre-cancerous condition is leukoplakia and the non-PCDGF based therapy is, e.g., Vitamin A (such as Accutane®, isotretinoin, 13-cis retinoic acid), beta-carotene, or antiviral medication (e.g., oral acyclovir, famciclovir, and zidovudine).

In another embodiment, the pre-cancerous condition is erythroplasia and the non-PCDGF based therapy is, e.g., 5-fluorouracil cream, etretinate, interferon-γ, or surgical removal

In another embodiment, the pre-cancerous condition is Bowen's disease and the non-PCDGF based therapy is, e.g., 5-fluorouracil cream, etretinate, interferon gamma freezing with liquid nitrogen, or surgical removal.

In another embodiment, the pre-cancerous condition is lymphomatoid papulosis and the non-PCDGF based therapy is, e.g., cortisone ointments, methotrexate, or ultraviolet light therapy.

In another embodiment, the pre-cancerous condition is adenomatous polyps and the non-PCDGF based therapy is, e.g., Celecoxib or surgical removal.

In other embodiments, the methods of the invention encompass administration of one or more PCDGF agents in combination with the administration of one or more therapeutic agents that are inhibitors of kinases such as, but not limited to, ABL, ACK, AFK, AKT (e.g., AKT-1, AKT-2, and AKT-3), ALK, AMP-PK, ATM, Auroral, Aurora2, bARK1, bArk2, BLK, BMX, BTK, CAK, CaM kinase, CDC2, CDK, CK, COT, CTD, DNA-PK, EGF-R, ErbB-1, ErbB-2, ErbB-3, ErbB-4, ERK (e.g., ERK1, ERK2, ERK3, ERK4, ERK5, ERK6, ERK7), ERT-PK, FAK, FGR (e.g., FGF1R, FGF2R), FLT (e.g., FLT-1, FLT-2, FLT-3, FLT-4), FRK, FYN, GSK (e.g., GSK1, GSK2, GSK3-alpha, GSK3-beta, GSK4, GSK5), G-protein coupled receptor kinases (GRKs), HCK, HER2, HKII, JAK (e.g., JAK1, JAK2, JAK3, JAK4), JNK (e.g., JNK1, JNK2, JNK3), KDR, KIT, IGF-1 receptor, IKK-1, IKK-2, INSR (insulin receptor), IRAK1, IRAK2, IRK, ITK, LCK, LOK, LYN, MAPK, MAPKAPK-1, MAPKAPK-2, MEK, MET, MFPK, MHCK, MLCK, MLK3, NEU, NIK, PDGF receptor alpha, PDGF receptor beta, PHK, PI-3 kinase, PKA, PKB, PKC, PKG, PRK1, PYK2, p38 kinases, p135tyk2, p34cdc2, p42cdc2, p42mapk, p44mpk, RAF, RET, RIP, RIP-2, RK, RON, RS kinase, SRC, SYK, S6K, TAK1, TEC, TIE1, TIE2, TRKA, TXK, TYK2, UL13, VEGFR1, VEGFR2, YES, YRK, ZAP-70, and all subtypes of these kinases (see e.g., Hardie and Hanks (1995) The Protein Kinase Pacts Book, I and II, Academic Press, San Diego, Calif.). In preferred embodiments, one or more PCDGF agents are administered in combination with the administration of one or more therapeutic agents that are inhibitors of Eph receptor kinases (e.g., EphA2 and EphA4). In a more preferred embodiment, one or more PCDGF agents are administered in combination with one or more anti-EphA2 antibodies Eph099B-102.147, Eph099B-208.261, Eph099B-210.248, Eph099B-233.152, EA2, and EA5. Hybridomas producing Eph099B-102.147, Eph099B-208.261, and Eph099B-210.248 have been deposited with the American Type Culture Collection (ATCC, P.O. Box 1549, Manassas, Va. 20108) on Aug. 7, 2002 under the provisions of the Budapest Treaty on the International Recognition of the Deposit of Microorganisms for the Purposes of Patent Procedures, and assigned accession numbers PTA-4572, PTA-4573, and PTA-4574, respectively, and incorporated by reference herein. A hybridoma producing Eph099B-233.152 has been deposited with the ATCC on May 12, 2003 under the provisions of the Budapest Treaty on the International Recognition of the Deposit of Microorganisms for the Purposes of Patent Procedures, and assigned accession number PTA-5194, and incorporated by reference herein (see co-pending U.S. patent application Ser. No. 10/436,782, entitled “EphA2 Monoclonal Antibodies and Methods of Use Thereof,” filed May 12, 2003). Hybridomas producing antibodies EA2 (strain EA2.31) and EA5 (strain EA5.12) have been deposited with the ATCC on May 22, 2002 under the provisions of the Budapest Treaty on the International Recognition of the Deposit of Microorganisms for the Purposes of Patent Procedures, and assigned accession numbers PTA-4380 and PTA-4381, respectively and incorporated by reference herein (see co-pending U.S. patent application Ser. No. 10/436,783, entitled “EphA2 Agonistic Monoclonal Antibodies and Methods of Use Thereof,” filed May 12, 2003 as Docket No. 10271-107-999).

Therapies for pre-cancerous conditions (e.g., chemotherapies, hormonal therapies, biological therapies/immunotherapies, radiation therapies) and their dosages, routes of administration and recommended usage are known in the art and have been described in such literature as the Physician's Desk Reference (56^{th} ed., 2002).

4.7. Identification of Agents of the Invention

The invention provides methods of assaying and screening for PCDGF agents by incubating candidate agents with cells that express or bind to a PCDGF polypeptide and then assaying for a desirable change in cell phenotype. Any cell that either expresses or responds to (e.g., expresses a PCDGF receptor) PCDGF can be used in the screening assays including pre-cancerous cells and cancer cells. Additionally, animal models of pre-cancerous conditions can be used to screen for PCDGF agents.

4.7.1. PCDGF Agents that Decrease Expression or Secretion

The invention provides methods of assaying and screening candidate agents for those agents that decrease PCDGF or PCDGF receptor expression, secretion, and/or activity. In one embodiment, a PCDGF agent decreases PCDGF or PCDGF receptor expression levels (e.g., decreases mRNA transcription or translation etc.). Any method known in the art for assaying PCDGF or PCDGF receptor expression can be used including, but not limited to, RT-PCR, northern blot analysis, western blot analysis, and ELISA.

In another embodiment, a PCDGF agent of the invention decreases/inhibits secretion of PCDGF or PCDGF receptor. Any method known in the art can be used to assay for candidate agents that decrease PCDGF or PCDGF receptor secretion. In a specific embodiment, conditioned medium from cells expressing PCDGF can be used for ELISA or western blot analysis or immunoprecipitation. In another specific embodiment, cells which express PCDGF receptor can be used for immunofluorescence or FACS analysis to assay if the PCDGF receptor extracellular domain is expressed on the surface of the cell. In a more specific embodiment, the PCDGF agent that decreases PCDGF or PCDGF receptor secretion is an intrabody.

4.7.2. PCDGF Agents that Decrease PCDGF-Receptor Binding

In another embodiment, the PCDGF agent inhibits/decreases binding of PCDGF to its receptor. In one embodiment, a PCDGF agent is a competitive inhibitor, non-competitive inhibitor, or uncompetitive inhibitor of PCDGF. In another embodiment, a PCDGF agent neutralizes PCDGF such that PCDGF cannot bind its receptor (see, e.g., Sections 4.1.2 and 4.1.3). In a specific embodiment, the PCDGF agent is a neutralizing antibody, preferably a monoclonal antibody. Such neutralizing antibodies can be utilized to generate anti-idiotype antibodies that “mimic” PCDGF polypeptides using techniques well known to those skilled in the art (see, e.g., Greenspan & Bona, 1993, FASEB 17:437-44; Nissinoff, 1991, J. Immunol. 147:2429-38). For example, PCDGF antibodies which bind to PCDGF and competitively inhibit the binding of PCDGF to its receptor can be used to generate anti-idiotypes that “mimic” the PCDGF ligand/receptor-binding domain and, as a consequence, bind to and neutralize PCDGF receptors. Such anti-idiotypic antibodies can be used to bind PCDGF ligands/receptors, and thereby block PCDGF-mediated biological activity. Alternatively, anti-idiotypes that “mimic” a PCDGF binding domain may bind to PCDGF receptors and block PCDGF from binding thus inhibiting receptor mediated signaling.

4.7.3. PCDGF Agents that Decrease Activity

In another embodiment, the PCDGF agent inhibits/decreases a biological effect normally observed when PCDGF binds its endogenous binding partner (e.g., receptor such as Rse). In a specific embodiment, the biological activity of PCDGF is increased cell proliferation. Many assays well-known in the art can be used to assess cell proliferation, such as, e.g., by measuring (³H)-thymidine incorporation, by direct cell count, by detecting changes in transcription, translation or activity of known genes such as cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as western blotting or immunoprecipitation using commercially available antibodies (for example, many cell cycle marker antibodies are from Santa Cruz Inc.). mRNA can be quantitated by methods that are well known and routine in the art, for example by northern analysis, RNase protection, the polymerase chain reaction in connection with reverse transcription (quantitative RT-PCR), in situ hybridization, etc.

The present invention provides for cell cycle and cell proliferation analysis by a variety of techniques known in the art, including but not limited to the following:

As one example, bromodeoxyuridine (BRDU) incorporation may be used as an assay to identify proliferating cells. The BRDU assay identifies a cell population undergoing DNA synthesis by incorporation of BRDU into newly synthesized DNA. Newly synthesized DNA may then be detected using an anti-BRDU antibody (see Hoshino et al., 1986, Int. J. Cancer 38:369; Campana et al., 1988, J. Immunol. Meth. 107:79).

Cell proliferation may also be examined using (³H)-thymidine incorporation (see e.g., Chen, 1996, Oncogene 13:1395-403; Jeoung, 1995, J. Biol. Chem. 270:18367-73). This assay allows for quantitative characterization of S-phase DNA synthesis. In this assay, cells synthesizing DNA will incorporate (³H)-thymidine into newly synthesized DNA. Incorporation may then be measured by standard techniques in the art such as by counting of radioisotope in a Scintillation counter (e.g. Beckman LS 3800 Liquid Scintillation Counter).

Detection of proliferating cell nuclear antigen (PCNA) may also be used to measure cell proliferation. PCNA is a 36 kilodalton protein whose expression is elevated in proliferating cells, particularly in early G1 and S phases of the cell cycle and therefore may serve as a marker for proliferating cells. Positive cells are identified by immunostaining using an anti-PCNA antibody (see Li et al., 1996, Curr. Biol. 6:189-99; Vassilev et al., 1995, J. Cell Sci. 108:1205-15).

Cell proliferation may be measured by counting samples of a cell population over time (e.g. daily cell counts). Cells may be counted using a hemacytometer and light microscopy (e.g. HyLite hemacytometer, Hausser Scientific). Cell number may be plotted against time in order to obtain a growth curve for the population of interest. In a preferred embodiment, cells counted by this method are first mixed with the dye Trypan-blue (Sigma), such that living cells exclude the dye, and are counted as viable members of the population.

DNA content and/or mitotic index of the cells may be measured, for example, based on the DNA ploidy value of the cell. For example, cells in the G1 phase of the cell cycle generally contain a 2N DNA ploidy value. Cells in which DNA has been replicated but have not progressed through mitosis (e.g. cells in S-phase) will exhibit a ploidy value higher than 2N and up to 4N DNA content. Ploidy value and cell-cycle kinetics may be further measured using propidum iodide assay (see e.g. Turner, et al., 1998, Prostate 34:175-81). Alternatively, the DNA ploidy may be determined by quantitation of DNA Feulgen staining (which binds to DNA in a stoichiometric manner) on a computerized microdensitometrystaining system (see e.g., Bacus, 1989, Am. J. Pathol. 135:783-92). In an another embodiment, DNA content may be analyzed by preparation of a chromosomal spread (Zabalou, 1994, Hereditas. 120:127-40; Pardue, 1994, Meth. Cell Biol. 44:333-351).

The expression of cell-cycle proteins (e.g., CycA. CycB, CycE, CycD, cdc2, Cdk4/6, Rb, p21, p27, etc.) provide crucial information relating to the proliferative state of a cell or population of cells. For example, identification in an anti-proliferation signaling pathway may be indicated by the induction of p21^{cip1}. Increased levels of p21 expression in cells results in delayed entry into G1 of the cell cycle (Harper et al., 1993, Cell 75:805-816; Li et al., 1996, Curr. Biol. 6:189-199). p21 induction may be identified by immunostaining using a specific anti-p21 antibody available commercially (e.g. Santa Cruz). Similarly, cell-cycle proteins may be examined by western blot analysis using commercially available antibodies. In another embodiment, cell populations are synchronized prior to detection of a cell cycle protein. Cell cycle proteins may also be detected by FACS (fluorescence-activated cell sorter) analysis using antibodies against the protein of interest.

PCDGF agents can also be identified by their ability to change the length of the cell cycle or speed of cell cycle so that cell proliferation is decreased or inhibited. In one embodiment the length of the cell cycle is determined by the doubling time of a population of cells (e.g., using cells contacted or not contacted with one or more candidate PCDGF agents). In another embodiment, FACS analysis is used to analyze the phase of cell cycle progression, or purify G1, S, and G2/M fractions (see, e.g., Delia et al., 1997, Oncogene 14:2137-47).

In another specific embodiment, the biological activity of PCDGF is activation of mitogen-activated protein (MAP) kinase, phosphatidylinosital 3′ kinase (PI3K), and/or focal adhesion kinase (FAK). Any method known in the art can be used to determine MAP, PI3K, or FAK activation.

In another specific embodiment, the biological activity of PCDGF is increased expression of cyclin D1, matrix metalloproteinase (MMP) 13, and/or MMP 17. Any method known in the art can be used to determine levels of cyclin D1, MMP13, or MMP17 including, but not limited to, RT-PCR, northern blot analysis, western blot analysis, and ELISA.

In another specific embodiment, the biological activity of PCDGF is increased phosphorylation of pRB. Any method known in the art can be used to determine phosphorylation levels of pRB. For example, cell lysates from cells incubated with PCDGF and a candidate agent can be immunoprecipitated with a pRB-specific antibody and then resolved by SDS-PAGE before being subjected to western blot analysis (see Taya et al., 2003, Methods Mol Biol. 223:17-26.).

4.7.4. PCDGF and PCDGF Receptor Antibodies with Low K_{off} Rates

In another embodiment, when the PCDGF agent is an antibody (preferably a monoclonal antibody), the PCDGF or PCDGF receptor antibody has a low K_{off} rate. The binding affinity of a antibody to its epitope (e.g., PCDGF, PCDGF receptor, or a fragment thereof) and the off-rate of a monoclonal antibody-epitope interaction can be determined by competitive binding assays. One example of a competitive binding assay is a radioimmunoassay comprising the incubation of labeled (e.g., ³H or ¹²⁵I) epitope (e.g., PCDGF, PCDGF receptor, or a fragment thereof) with the antibody of interest in the presence of increasing amounts of unlabeled epitope, and the detection of the monoclonal antibody bound to the labeled epitope. The affinty of a monoclonal antibody for its epitope and the binding off-rates can be determined from the data by scatchard plot analysis. Competition with a second antibody can also be determined using radioimmunoassays. In this case, epitope (e.g., PCDGF, PCDGF receptor, or a fragment thereof) is incubated with a monoclonal antibody conjugated to a labeled compound (e.g., ³H or ¹²⁵I) in the presence of increasing amounts of a second unlabeled antibody.

In a preferred embodiment, BIACORE™ kinetic analysis is used to determine the binding on and off rates of antibodies to their epitopes (e.g., PCDGF, PCDGF receptor, or a fragment thereof). BIACORE™ kinetic analysis comprises analyzing the binding and dissociation of a monoclonal antibody from chips with immobilized epitopes (e.g., PCDGF, PCDGF receptor, or a fragment thereof) on their surface.

In some embodiments, an antibody that immunospecifically binds PCDGF or PCDGF receptor preferably has a K_{off} rate (antibody (Ab)+antigen (Ag) [symbol] Ab-Ag) of less than less than 10⁻³ s⁻¹, less than, less than 9×10⁻⁴ s⁻¹, less than 8×10⁻⁴ s⁻¹, less than 7×10⁻⁴ s⁻¹, less than 5×10⁻⁴ s⁻¹, less than 10⁻⁴ s⁻¹, less than 9×10⁻⁵ s⁻¹, less than 5×10⁻⁵ s⁻¹, less than 10⁻⁵ s⁻¹, less than 5×10⁻⁶ s⁻¹, less than 10⁻⁶ s⁻¹, less than 5×10⁻⁷ s⁻¹, less than 10⁻⁷ s⁻¹, less than 5×10⁻⁸ s⁻¹, less than 10⁻⁸ s⁻¹, less than 5×10⁻⁹ s⁻¹, less than 10⁻⁹ s⁻¹, or less than 10⁻¹⁰ s⁻¹. In more specific embodiments, an antibody that immunospecifically binds PCDGF preferably has a K_{off} rate between 5×10⁻⁴ s⁻¹ and 8×10⁻⁴ s⁻¹.

4.8. Characterization and Demonstration of Therapeutic Utility

Toxicity and efficacy of the therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapeutic agents that exhibit large therapeutic indices are preferred. While therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The anti-pre-cancerous activity of the therapies used in accordance with the present invention also can be determined by using various experimental animal models for the study of pre-cancerous conditions such as the SCID mouse model or transgenic mice where a mouse gene of interest (e.g., PCDGF or PCDGF receptor) is replaced with the corresponding human gene or portion thereof, nude mice with human xenografts, animal models described in Section 5 infra, or any animal model (including hamsters, rabbits, etc.) known in the art and described in Relevance of Tumor Models for Anticancer Drug Development (1999, eds. Fiebig and Burger); Contributions to Oncology (1999, Karger); The Nude Mouse in Oncology Research (1991, eds. Boven and Winograd); and Anticancer Drug Development Guide (1997 ed. Teicher), incorporated by reference in their entireties herein.

4.9. Demonstration of Therapeutic Utility

The protocols and compositions of the invention are preferably tested in vitro, and then in vivo, for the desired therapeutic activity, prior to use in humans. For example, in vitro assays which can be used to determine whether administration of a specific therapeutic protocol is indicated, include in vitro cell culture assays in which a patient tissue sample is grown in culture, and exposed to or otherwise administered a protocol, and the effect of such protocol upon the tissue sample is observed, e.g., decreased expression, secretion, and/or activity of the PCDGF and/or PCDGF receptor polypeptide. A lower level of proliferation, MAP activation, PI3K activation, FAK activation, cyclin D1 expression, MMP13 expression, MMP17 expression, phosphorylation of pRB, and/or progression to cancer of the contacted cells indicates that the therapeutic agent is effective to treat the condition in the patient. Alternatively, instead of culturing cells from a patient, therapeutic agents and methods may be screened using cells of a cell line.

Agents for use in therapy can be tested in suitable animal model systems prior to testing in humans, including but not limited to in rats, mice, chicken, cows, monkeys, rabbits, hamsters, etc., for example, the animal models described above. The agents can then be used in the appropriate clinical trials.

Further, any assays known to those skilled in the art can be used to evaluate the therapeutic utility of the therapy disclosed herein for treatment or management of a pre-cancerous condition or cancer and/or the delay or decrease in the likelihood that the pre-cancerous condition will progress to cancer.

4.10. Pharmaceutical Compositions

The compositions of the invention include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a therapeutically effective amount of a therapeutic agent disclosed herein or a combination of those agents and a pharmaceutically acceptable carrier. Preferably, compositions of the invention comprise a therapeutically effective amount of one or more one or more PCDGF agents and a pharmaceutically acceptable carrier. In a further embodiment, the composition of the invention further comprises an additional cancer therapeutic that is not PCDGF-based.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete) or, more preferably, MF59C.1 adjuvant available from Chiron, Emeryville, Calif.), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Various delivery systems are known and can be used to administer a therapeutic agent useful for treating or managing a pre-cancerous condition, e.g., encapsulation in liposomes, microparticles, microcapsules, recombinant cells capable of expressing the polypeptide fragment, receptor-mediated endocytosis (see, e.g., Wu and Wu, 1987, J. Biol. Chem. 262:4429-4432), construction of a nucleic acid as part of a retroviral or other vector, etc. Methods of administering a therapeutic agent include, but are not limited to, parenteral administration (e.g., intradermal, intramuscular, intraperitoneal, intravenous and subcutaneous), epidural, and mucosal (e.g., intranasal, inhaled, and oral routes). In a specific embodiment, therapeutic agents of the invention are administered intramuscularly, intravenously, or subcutaneously. The therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the therapeutic agents for use in methods of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

In yet another embodiment, the therapeutic agent can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the agents of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; International Publication Nos. WO 99/15154 and WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938; International Publication Nos. WO 91/05548 and WO 96/20698; Ning et al., 1996, Radiotherapy &Oncology 39:179-189; Song et al., 1995, PDA Journal of Pharmaceutical Science &Technology 50:372-397; Cleek et al., 1997, Proc. Int'l. Symp. Control. Rel. Bioact. Mater. 24:853-854; and Lam et al., 1997, Proc. Int'l. Symp. Control Rel. Bioact. Mater. 24:759-760.

4.10.1. Gene Therapy

In a specific embodiment, nucleic acids (e.g., antisense nucleic acids specific for PCDGF, double stranded PCDGF RNA that mediates RNAi, anti-PCDGF ribozymes, nucleotide encoding a PCDGF intrabody, antisense nucleic acids specific for PCDGF receptor, double stranded PCDGF receptor RNA that mediates RNAi, anti-PCDGF receptor ribozymes, nucleotide encoding a PCDGF receptor intrabody etc.) that reduce expression of a PCDGF or PCDGF receptor polypeptide are administered to treat or manage a pre-cancerous condition by way of gene therapy. Gene therapy refers to therapy performed by the administration to a subject of an expressed or expressible nucleic acid. In this embodiment of the invention, the nucleic acids are produced and mediate therapeutic effect (either directly or indirectly after translation).

Any of the methods for gene therapy available in the art can be used according to the present invention. Exemplary methods are described below.

For general reviews of the methods of gene therapy, see Goldspiel et al., 1993, Clinical Pharmacy 12:488; Wu and Wu, 1991, Biotherapy 3:87; Tolstoshev, 1993, Ann. Rev. Pharmacol. Toxicol. 32:573; Mulligan, 1993, Science 260:926-932; and Morgan and Anderson, 1993, Ann. Rev. Biochem. 62:191; May, 1993, TIBTECH 11:155. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, a composition of the invention comprises nucleic acid agents for use in the methods of the invention, said nucleic acids being part of an expression vector that expresses the nucleic acid in a suitable host. In particular, such nucleic acids have promoters, preferably heterologous promoters, said promoter being inducible or constitutive, and, optionally, tissue-specific. In another particular embodiment, nucleic acid molecules are used in which the nucleic acid agent is flanked by regions that promote homologous recombination at a desired site in the genome, thus providing for intrachromosomal expression of the nucleic acids that reduce PCDGF or PCDGF receptor expression (Koller and Smithies, 1989, PNAS 86:8932; Zijlstra et al., 1989, Nature 342:435).

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy. In a specific embodiment, the nucleic acid sequences are directly administered in vivo. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., International Publication Nos. WO 92/06180; WO 92/22635; WO92/203 16; WO93/14188, WO 93/20221). Alternatively, the nucleic acid can be introduced intracellularly and incorporated within host cell DNA for expression, by homologous recombination (Koller and Smithies, 1989, PNAS 86:8932; and Zijlstra et al., 1989, Nature 342:435).

In a specific embodiment, viral vectors that contain the nucleic acid sequences that reduce PCDGF expression are used. For example, a retroviral vector can be used (see Miller et al., 1993, Meth. Enzymol. 217:581). These retroviral vectors contain the components necessary for the correct packaging of the viral genome and integration into the host cell DNA. The nucleic acid sequences to be used in gene therapy are cloned into one or more vectors, which facilitates delivery of the nucleic acid into a subject. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Adenoviruses have the advantage of being capable of infecting non-dividing cells. Kozarsky and Wilson, 1993, Current Opinion in Genetics Development 3:499 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431; Rosenfeld et al., 1992, Cell 68:143; Mastrangeli et al., 1993, J. Clin. Invest. 91:225; International Publication No. WO94/12649; and Wang et al., 1995, Gene Therapy 2:775. In a preferred embodiment, adenovirus vectors are used.

Adeno-associated virus (AAV) has also been proposed for use in gene therapy (Walsh et al., 1993, Proc. Soc. Exp. Biol. Med. 204:289-300; and U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell mediated gene transfer, spheroplast fusion, etc. Numerous techniques are known in the art for the introduction of foreign genes into cells (see, e.g., Loeffler and Behr, 1993, Meth. Enzymol. 217:599; Cohen et al., 1993, Meth. Enzymol. 217:618) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. The amount of cells envisioned for use depends on the desired effect, patient state, etc., and can be determined by one skilled in the art.

4.10.2. Formulations

Pharmaceutical compositions for use in accordance with the present invention may be formulated in conventional manner using one or more physiologically acceptable carriers or excipients.

Thus, the agents for use in the methods of the invention and their physiologically acceptable salts and solvates may be formulated for administration by inhalation or insufflation (either through the mouth or the nose) or oral, parenteral or mucosal (such as buccal, vaginal, rectal, sublingual) administration. In a preferred embodiment, local or systemic parenteral administration is used.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the therapeutic agents for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The therapeutic agents may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The therapeutic agents may also be formulated in rectal compositions such as suppositories or retention enemas, e.g., containing conventional suppository bases such as cocoa butter or other glycerides.

In addition to the formulations described previously, the therapeutic agents may also be formulated as a depot preparation. Such long acting formulations may be administered by implantation (for example subcutaneously or intramuscularly) or by intramuscular injection. Thus, for example, the therapeutic agents may be formulated with suitable polymeric or hydrophobic materials (for example as an emulsion in an acceptable oil) or ion exchange resins, or as sparingly soluble derivatives, for example, as a sparingly soluble salt.

The invention also provides that a therapeutic agent is packaged in a hermetically sealed container such as an ampoule or sachette indicating the quantity. In one embodiment, the therapeutic agent is supplied as a dry sterilized lyophilized powder or water free concentrate in a hermetically sealed container and can be reconstituted, e.g., with water or saline to the appropriate concentration for administration to a subject.

In a preferred embodiment of the invention, the formulation and administration of various therapies for pre-cancerous conditions that are not PCDGF-based (such as chemotherapeutic, biological/immunotherapeutic and hormonal therapeutic agents) are known in the art and often described in the Physician's Desk Reference, 56^{th} ed. (2002).

In other embodiments of the invention, radiation therapy agents such as radioactive isotopes can be given orally as liquids in capsules or as a drink. Radioactive isotopes can also be formulated for intravenous injections. The skilled oncologist can determine the preferred formulation and route of administration.

In certain embodiments the agents of the invention, are formulated at 1 mg/ml, 5 mg/ml, 10 mg/ml, and 25 mg/ml for intravenous injections and at 5 mg/ml, 10 mg/ml, and 80 mg/ml for repeated subcutaneous administration and intramuscular injection.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

4.10.3. Dosages

The amount of the composition of the invention which will be effective in the treatment or management of a pre-cancerous condition can be determined by standard research techniques. For example, the dosage of the composition which will be effective in the treatment or management of a pre-cancerous condition can be determined by administering the composition to an animal model such as, e.g., the animal models disclosed herein or known to those skilled in the art. In addition, in vitro assays may optionally be employed to help identify optimal dosage ranges.

Selection of the preferred effective dose can be determined (e.g., via clinical trials) by a skilled artisan based upon the consideration of several factors which will be known to one of ordinary skill in the art. Such factors include the disease to be treated or prevented, the symptoms involved, the patient's body mass, the patient's immune status and other factors known by the skilled artisan to reflect the accuracy of administered pharmaceutical compositions.

The precise dose to be employed in the formulation will also depend on the route of administration, and the seriousness of the cancer, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human and humanized antibodies have a longer half-life within the human body than antibodies from other species due to the immune response to the foreign polypeptides. Thus, lower dosages of human antibodies and less frequent administration is often possible.

For therapeutic agents that are not PCDGF-based, the typical doses of various therapeutics for pre-cancerous conditions are known in the art. Given the invention, certain preferred embodiments will encompass the administration of lower dosages in combination treatment regimens than dosages recommended for the administration of single agents.

The invention provides for any method of administrating lower doses of known therapeutic agents than previously thought to be effective for the treatment or management or amelioration of a pre-cancerous condition. Preferably, lower doses of known therapies are administered in combination with PCDGF agents.

4.11. Kits

The invention provides a pharmaceutical pack or kit comprising one or more containers filled with one or more PCDGF agents. Additionally, one or more therapeutic agents useful for the treatment of a pre-cancerous condition that PCDGF-based can also be included in the pharmaceutical pack or kit. In a specific embodiment, the non-PCDGF-based agent decreases the expression and/or activity of EphA2 (e.g., anti-EphA2 monoclonal antibodies Eph099B-102.147, Eph099B-208.261, Eph099B-210.248, Eph099B-233.152, EA2, or EA5). The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

4.12. Diagnosis of Pre-Cancerous Conditions

In an alternative embodiment, the PCDGF agents of the invention can also be used in diagnostic assays either in vivo or in vitro for detection and/or identification of a pre-cancerous condition in a subject or a biological sample (e.g., cells or tissue). In a preferred embodiment, a PCDGF agent of the invention is a PCDGF or a PCDGF receptor antibody (see, e.g., Sections 4.1.1.1 and 4.1.1.2, discussed supra). Another preferred embodiment provides a method for diagnosing a pre-cancerous condition comprising contacting the cells in a subject suspected of having a pre-cancerous condition or a biological sample from said subject with a PCDGF antibody or a PCDGF receptor antibody under conditions appropriate for antibody binding, wherein a higher level of PCDGF antibody- or PCDGF receptor antibody-binding as compared to the level in a control subject that does not have a pre-cancerous condition, or sample therefrom, indicates that the subject has a pre-cancerous condition. In particular embodiments, the diagnostic methods of the invention provide methods of imaging and localizing pre-cancer cells in tissues and fluids, for example, whole blood, sputum, urine, serum, fine needle aspirates (i.e., biopsies). The PCDGF antibodies and/or PCDGF receptor antibodies of the invention may also be used for immunohistochemical analyses of frozen or fixed cells or tissue assays (e.g., immunohistochemical staining) using any standard method known to one skilled in the art. Non-limiting examples of using an antibody, or fragment thereof, or a composition comprising an antibody or a fragment thereof in a diagnostic assay are given in U.S. Pat. Nos. 6,392,020; 6,156,498; 6,136,526; 6,048,528; 6,015,555; 5,833,988; 5,811,310; 5,652,114; 5,604,126; 5,484,704; 5,346,687; 5,318,892; 5,273,743; 5,182,107; 5,122,447; 5,080,883; 5,057,313; 4,910,133; 4,816,402; 4,742,000; 4,724,213; 4,724,212; 4,624,846; 4,623,627; 4,618,486; 4,176,174 (all of which are incorporated by reference herein). Suitable diagnostic assays for the antigen (e.g., PCDGF or PCDGF receptor) and its antibodies depend on the particular antibody used. Non-limiting examples are an ELISA, sandwich assay, and steric inhibition assays. For in vivo diagnostic assays using the antibodies of the invention, the antibodies may be conjugated to a label that can be detected by imaging techniques, such as X-ray, computed tomography (CT), ultrasound, or magnetic resonance imaging (MRI). The antibodies of the invention can also be used for the affinity purification of the antigen from recombinant cell culture or natural sources.

In yet further embodiments, the invention provides methods for diagnosing/detecting a pre-cancerous condition by providing methods for detecting altered (e.g., increased) levels of PCDGF or PCDGF receptor expression in a subject suspected of having a pre-cancerous condition, or a biological sample from said subject, by measuring PCDGF or PCDGF receptor mRNA levels. In a preferred embodiment, a method for diagnosing a pre-cancerous condition comprises measuring the level of PCDGF mRNA or PCDGF receptor mRNA in a subject suspected of having a pre-cancerous condition, or a biological sample from said subject, wherein the level of PCDGF or PCDGF receptor mRNA is increased in said subject relative to a control subject that does not have a pre-cancerous condition, or biological sample therefrom. PCDGF or PCDGF receptor mRNA can be detected and/or quantitated by methods that are well known and routine in the art, for example, by northern analysis, RNase protection, the polymerase chain reaction in connection with reverse transcription (quantitative RT-PCR), in situ hybridization, etc.

### 5. EXAMPLES

5.1. Expression of PCDGF in Prostatic Intraepithelial Neoplasia

PCDGF immunoreactivity distinguished non-cancerous, normal prostate tissue from pre-cancerous (PIN) or neoplastic prostatic epithelial cells. Ninety nine cases of radical retropubic prostatectomy were obtained from the surgical pathology files of Indiana University Medical Center (Table 6). Patients ranged in age from 44 to 77 years (mean=63 years). Grading of the primary tumor from radical prostatectomy specimens was performed according to the Gleason system (Bostwick “Neoplasms of the prostate” in Bostwick and Eble, eds., 1997, Urologic Surgical Pathology St. Louis:Mosby page 343-422; Gleson and Mellinger, 1974, J. Urol. 111:58-64). The Gleason grade ranged from 4 to 10. Pathological stage was evaluated according to the 1997 TNM (tumor, lymph nodes, and metastasis) standard (Fleming et al., 1997, AJCC Cancer Staging Manual. Philadelphia:Raven and Lippincott). At the time of surgery, 14.1% of the patients had positive lymph node metastasis and 29.3% had vascular invasion.

Serial 5 μm-thick sections of formalin-fixed slices of radical prostatectomy specimens were used for immunofluorescent staining. Tissue blocks that contained the maximum amount of tumor and highest Gleason grade were selected. One representative slide from each case was analyzed. Slides were deparaffinized in xylene twice for 5 minutes and rehydrated through graded ethanols to distilled water. Antigen retrieval was carried out by heating sections in EDTA (pH 8.0) for 30 minutes. Endogenous peroxidase activity was inactivated by incubation in 3% H₂O₂ for 15 minutes. Non-specific binding sites were blocked using Protein Block (DAKO Corporation, Carpintera, Calif.) for 20 minutes. Tissue sections were then incubated with a rabbit polyclonal antibody against human PCDGF (6B2, 1:200 dilution, A&G Pharmaceuticals, Inc. Columbia, Md.) overnight at room temperature, followed by biotinylated secondary antibody (DAKO corporation) and peroxidase-labeled streptavidin. 3,3-diaminobenzidine was used as the chromogen in the presence of hydrogen peroxide. Positive and negative controls were run in parallel with each batch.

The extent and intensity of staining were evaluated in benign epithelium, high-grade prostatic intraepithelial neoplasia (PIN) and adenocarcinoma from the same slide for each case. Microscopic fields with highest degree of immunoreactivity were chosen for analysis. At least 1000 cells were analyzed in each case. The percentage of cells exhibiting staining in each case was evaluated semiquantitatively on a 5% incremental scale ranging from 0 to 95%. A numeric intensity score was set from 0 to 3 (0=no staining; 1=weak staining; 2=moderate staining; and 3=strong staining) (Jiang et al., 2002, Am. J. Pathol. 160:667-71; Cheng et al., 1996, Am J. Pathol. 148:1375-80).

The mean percentage of immunoreactive cells in benign epithelium, high-grade PIN and adenocarcinoma were compared using the Wilcoxon paired signed rank test. The intensity of staining for PCDGF in benign epithelium, high-grade PIN, and adenocarcinoma was compared using Cochran-Mantel-Haenszel tests for correlated ordered categorical data. A p-value<0.05 was considered significant, and all p-values were two-sided.

PCDGF was not expressed or was detected at low levels in histologically normal (non-cancerous, non-pre-cancerous) cells within cancer tissues (Tables 4 and 5). Initially, immunoreactivity against non-cancerous cells was scored as a 0 (in 54% of specimens) or 1 (in 46% of specimens) when using a 0-3 scale. A more detailed analysis of the PCDGF-positive samples revealed that overall, fewer than 5% of the non-cancerous, non-pre-cancerous cells within these specimens demonstrated PCDGF immunoreactivity.

PCDGF was expressed at high levels in both pre-cancerous (PIN) and invasive prostate cancer tissue. All (99 of 99) PIN and (99 of 99) invasive cancer specimens demonstrated intermediate or high levels of PCDGF immunoreactivity (staining intensity of 2 or 3) (Tables 4 and 5). Intermediate staining (intensity=2) was observed in 51% of PIN and 45% of invasive cancer tissue whereas strong PCDGF staining (intensity=3) was observed in 49% of PIN and 55% of invasive cancer tissue. In all PIN and cancer tissue, at least 50% of the diseased cells reacted with PCDGF antibodies. Furthermore, PCDGF stained more than 90% of diseased cells in 56% of PIN and 78% of invasive prostate cancer cells. Consequently, the mean percentage of diseased cells that reacted with PCDGF antibodies averaged 84% for PIN and 90% for invasive cancer. When compared with normal (non-cancerous, non-pre-cancerous) prostate tissue, both the intensity and the fraction of cells expressing PCDGF were significantly elevated in PIN and invasive cancer (P<0.0001). However, PCDGF immunoreactivity in PIN and invasive cancer was not statistically significant (P=0.10) and reflected the fact that PCDGF appears to be upregulated prior to accumulation of cellular defects that characterize PIN.

There was significant concordance of PCDGF expression between PIN and invasive cancer. Most of specimens (42/50 specimens) where PIN cells had high levels of PCDGF expression also displayed high PCDGF expression in the invasive cancer cells. Similarly, most of the specimens (38/47 specimens) where the invasive cancer cells expressed intermediate levels of PCDGF (staining intensity of 2) also displayed PIN cells with lower PCDGF expression. Although high levels of PCDGF could distinguish pre-cancerous and neoplastic tissue from benign (non-cancerous, non-pre-cancerous) prostatic epithelial cells, PCDGF did not correlate with other histologic and pathologic parameters of disease severity. For example, high levels of PCDGF were observed in most invasive cancer cells but did not relate to Gleason grade, pathologic stage, lymph node metastasis, extraprostatic extension, surgical margins, vascular invasion, perineural invasion or the presence of other areas of the same prostate with high-grade PIN (Table 6).

TABLE 4

| | Staining Intensity Grade | | | |
|---|---|---|---|---|
| Tissue Type | 0 | 1 | 2 | 3 |
| Benign epithelium | 55 (54%) | 46 (46%) | 0 | 0 |
| High-grade PIN | 0 | 1 (1%) | 51 (52%) | 47 (48%) |
| Invasive Cancer | 0 | 0 | 45 (46%). | 54 (54%) |

TABLE 5

| Tissue | Staining (%) | Mean Staining ± SD | Range |
|---|---|---|---|
| Benign epithelium | 49 | 5.3 ± 7.5 | 0-40 |
| High-grade PIN | 99 | 84.2 ± 12.6^{a} | 50-95 |
| Invasive Cancer | 99 | 90.3 ± 8.3 | 50-95 |

TABLE 6

| | | % of Total | Mean % of Cells | Mean PCDGF |
|---|---|---|---|---|
| | | Patients | Staining w/ PCDGF | Antibody Staining |
| Patient Characteristic | | (n = 99) | Antibody (±SD) | Intensity (±SD) |
| Primary Gleason Grade | 2 | 13 | 91 ± 7 | 2.5 ± 0.5 |
| | 3 | 46 | 90 ± 10 | 2.6 ± 0.5 |
| | 4 | 27 | 90 ± 8 | 2.4 ± 0.5 |
| | 5 | 13 | 91 ± 8 | 2.6 ± 0.5 |
| Secondary Gleason Grade | 2 | 15 | 89 ± 8 | 2.4 ± 0.5 |
| | 3 | 35 | 91 ± 7 | 2.5 ± 0.5 |
| | 4 | 35 | 88 ± 11 | 2.6 ± 0.5 |
| | 5 | 14 | 93 ± 5 | 2.6 ± 0.5 |
| Gleason Sum | <7 | 30 | 91 ± 7 | 2.5 ± 0.5 |
| | 7 | 41 | 88 ± 11 | 2.6 ± 0.5 |
| | >7 | 28 | 91 ± 6 | 2.6 ± 0.5 |
| T Classification | T2a | 11 | 91 ± 7 | 2.5 ± 0.5 |
| | T2b | 46 | 90 ± 8 | 2.6 ± 0.5 |
| | T3a | 26 | 90 ± 10 | 2.4 ± 0.5 |
| | T3b | 16 | 90 ± 7 | 2.5 ± 0.5 |
| Lymph Node Metastasis | Positive | 14 | 88 ± 13 | 2.5 ± 0.5 |
| | Negative | 85 | 90 ± 8 | 2.3 ± 0.5 |
| Extraprostatic Extension | Positive | 59 | 90 ± 8 | 2.6 ± 0.5 |
| | Negative | 40 | 90 ± 9 | 2.5 ± 0.5 |
| Surgical Margin | Positive | 58 | 90 ± 7 | 2.5 ± 0.5 |
| | Negative | 41 | 90 ± 10 | 2.6 ± 0.5 |
| Vascular Invasion | Positive | 29 | 90 ± 9 | 2.4 ± 0.5 |
| | Negative | 70 | 90 ± 8 | 2.6 ± 0.5 |
| Perineural Invasion | Positive | 86 | 90 ± 9 | 2.5 ± 0.5 |
| | Negative | 13 | 90 ± 7 | 2.5 ± 0.5 |
| High-grade PIN | Positive | 95 | 81 ± 12 | 2.3 ± 0.6 |
| | Negative | 4 | 90 ± 8 | 2.5 ± 0.5 |

5.2. Preparation of Monoclonal Antibodies

Immunization and Fusion

Monoclonal antibodies against PCDGF are generated using recombinant PCDGF protein.

Two groups of mice (either Balb/c mice or SJL mice) are injected with 5 μg of PCDGF in TiterMax Adjuvant (total volume 100 μl) in the left metatarsal region at days 0 and 7. Mice are injected with 10 μg of PCDGF in PBS (total volume 100 μl) in the left metatarsal region at days 12 and 14. On day 15, the popliteal and inguinal lymph nodes from the left leg and groin are removed and somatically fused (using PEG) with P3XBcl-2-13 cells.

Antibody Screening

Supernatants from bulk culture hybridomas are screened for immunoreactivity against PCDGF using standard molecular biological techniques (e.g., ELISA immunoassay). Supernatants are further screened for the ability to inhibit PCDGF from binding to its receptor or causing a response in PCDGF-responsive cells.

5.3. Kinetic Analysis of PCDGF Antibodies

The BIACORE™ assay is used to measure the K_{off} rates of PCDGF monoclonal antibodies. IgG present in hybridoma supernatant is used for measurement.

Immobilization of PCDGF

PCDGF is immobilize to a surface on a CM5 sensorchip using a standard amine (70 μl of a 1:1 mix of NHS/EDC) coupling chemistry. Briefly, a 400 nM solution of PCDGF in 10 mM NaOAc, pH4, is injected over the activated surface to a density of 1000-1100 RU's. Unused reactive esters are subsequently “capped” with a 70 μl injection of 1M Et-NH2. Similarly, an activated and “capped” control surface is prepared on the same sensor chip without protein to serve as a reference surface.

Binding Experiments

A 250 μl injection of PCDGF monoclonal antibody hybridoma supernatant is made over both the PCDGF and control surfaces, and the binding responses are recorded. Following each injection, at least 10 min. of dissociation phase data is collected. A negative control monoclonal antibody that does not bind PCDGF is also prepared at 5 μg/250 μl growth medium. Control injections of growth medium across these surfaces are also made. Following each binding cycle, the PCDGF surface is regenerated with a single 1 min. pulse (injection) of 1M NaCl-50 mM NaOH.

Data Evaluation

The binding data is corrected by subtracting out both artifactual noise (blank medium injections) and non-specific binding (control surface), in a technique known as “double-referencing.” Thus the sensorgram overlays represent “net” binding curves.

5.4. Decreased PCDGF Levels Using PCDGF Antisense Oligonucleotides

PCDGF expression is reduced using an antisense oligonucleotide-based approach. To decrease PCDGF protein levels, PCDGF expressing cells are transiently transfected with phosphorothioate-modified antisense oligonucleotides that correspond to a sequence that is found to be unique to PCDGF as determined using a sequence evaluation of Genbank (e.g., 5′-GGG TCC ACA TGG TCT GCC TGC-3′ (SEQ ID NO:43) or 5′-GCC ACC AGC CCT GCT GTT AAG GCC-3′ (SEQ ID NO:44)). Inverted antisense oligonucleotides provides a control. The cells are transfected with oligonucleotides (2 μg/ml) using Lipofectamine PLUS Reagent (Life Technologies, Inc.) according to the manufacturer's protocol. Twenty-four hours post-transfection, the cells are extracted and subjected to western blot analysis.

Western blot analyses and immunoprecipitations are performed as described previously (Zantek et al., 1999, Cell Growth Diff. 10:629-38). Briefly, detergent extracts of cells are extracted in Tris-buttered saline containing 1% Triton X-100 (Sigma, St. Louis, Mo.). After measuring protein concentrations (BioRad, Hercules, Calif.), 1.5 mg of cell lysate is immunoprecipitated, resolved by SDS-PAGE and transferred to nitrocellulose (Protran, Schleicher and Schuell, Keene, N.H.). PCDGF is detected with a PCDGF-specific antibody (e.g., 6B2). To control for sample loading, the membranes are stripped and re-probed with paxillin antibodies. Antibody binding is detected by enhanced chemiluminescence (Pierce, Rockford, Ill.) and autoradiography (Kodak X-OMAT; Rochester, N.Y.).

5.5. Treatment of Patients with PIN

A study is designed to assess pharmacokinetics and safety of PCDGF agents in patients with PIN. Patients currently receiving treatment are permitted to continue these medications.

Patients are administered a single IV dose of a PCDGF agent. Four weeks later, the patients are analyzed following administration of repeated weekly IV doses of the therapy at the same dose over a period of 12 weeks. The safety of treatment with the PCDGF agent is assessed as well as potential changes in pre-cancerous activity over 26 weeks of IV dosing. Different groups of patients are treated and evaluated similarly but receive doses of 1 mg/kg, 2 mg/kg, 4 mg/kg, or 8 mg/kg.

PCDGF agents are formulated at 5 mg/ml and 10 mg/ml for IV injection. A formulation of 80 mg/ml is required for repeated subcutaneous administration. The PCDGF agents are also formulated at 100 mg/ml for administration for the purposes of the study.

Changes are measured or determined by the progression of PIN to prostate cancer (e.g., by PSA levels).

6. Equivalents

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

We claim:

1. A method of diagnosing, prognosing or monitoring the efficacy of a therapy to prevent or delay the progression of a pre-cancerous condition to cancer in a subject known to or suspected to have a pre-cancerous condition, said method comprising:
a) contacting cells of said subject with a PCDGF antibody under conditions appropriate for antibody binding; and
b) detecting said PCDGF antibody and binding to said cells,
wherein detecting a higher level of binding of said PCDGF antibody than the level of binding of said PDGF antibody in cells of a control subject that does not have a pre-cancerous condition indicates that said subject has a pre-cancerous condition.

2. The method of claim 1, wherein said cells are from whole blood, sputum, urine, serum or fine needle aspirates of pre-cancerous tissue.

3. The method of claim 2, wherein said cells are in frozen or fixed tissue or cells from said subject.

4. A method of detecting or diagnosing a pre-cancerous condition in a subject suspected of having a pre-cancerous condition, wherein said method comprises detecting the presence of PCDGF or PCDGF receptor in the cells of said subject, or a biological sample therefrom, using a PCDGF agent.

5. The method of claim 4, wherein said PCDGF agent is an anti-PCDGF antibody or an anti-PCDGF receptor antibody.

6. The method of claim 5, wherein said anti-PCDGF antibody or anti-PCDGF receptor antibody is human or humanized.

7. The method of claim 5, wherein said method comprises immunohistochemical staining using said anti-PCDGF antibody or anti-PCDGF receptor antibody.

8. The method of claim 7, wherein a detection of a higher level of antibody binding to PCDGF or PCDGF receptor in the cells of said subject, or a biological sample therefrom, relative to the cells in a control subject, or a biological sample therefrom, that does not have a pre-cancerous condition, indicates that said subject has a pre-cancerous condition.

9. The method of claim 4, wherein said cells are from whole blood, sputum, urine, serum or fine needle aspirates of pre-cancerous tissue.

10. The method of claim 4, wherein said cells are in frozen or fixed tissue or cells from said subject.

11. The method of claim 10, wherein said tissue or cells are from the breast, cervix, colon, esophagus, liver, lung, pancreas, prostate, skin, or stomach of said subject.

12. The method of claim 4, wherein said pre-cancerous condition is a condition of the breast, cervix, colon, esophagus, liver, lung, pancreas, prostate, skin, or stomach.

13. The method of claim 12, wherein said pre-cancerous condition of the breast is ductal carcinoma in situ (DCIS), fibrocystic disease, fibroadenoma of the breast, lobular carcinoma in situ, or intraductal hyperplasia.

14. The method of claim 12, wherein said pre-cancerous condition of the cervix is cervix dysplasia or squamous intraepithelial lesions (SIL).

15. The method of claim 12, wherein said pre-cancerous condition of the colon is adenomatous polyps.

16. The method of claim 12, wherein said pre-cancerous condition of the esophagus is Barrett's esophageal dysplasia.

17. The method of claim 12, wherein said pre-cancerous condition of the liver is hepatocellular carcinoma or adenomatous hyperplasia.

18. The method of claim 12, wherein said pre-cancerous condition of the lung is atypical adenomatous hyperplasia (AAH) of the lung, lymphoma, or lymphomatoid granulomatosis.

19. The method of claim 12, wherein said pre-cancerous condition of the pancreas is pancreatic ductal lesion, pancreatic hyperplasia, or pancreatic dysplasia.

20. The method of claim 12, wherein said pre-cancerous condition of the prostate is prostatic intraepithelial neoplasia (PIN).

21. The method of claim 12, wherein said pre-cancerous condition of the skin is xeroderma pigmentosum, carcinoma in situ of the skin, squamous cell carcinoma, solar keratosis, compound nevi, dysplastic nevi, actinic cheilitis, leukoplakia, erythroplasia, Bowen's disease, or lymphomatoid papulosis.

22. The method of claim 12, wherein said pre-cancerous condition of the stomach is adenomatous polyps.

23. The method of claim 4, wherein said pre-cancerous condition comprises cells that overexpress PCDGF relative to non-pre-cancerous cells having the tissue type of said pre-cancerous cells.

24. The method of claim 4, wherein said pre-cancerous condition comprises cells that are hyper-responsive to PCDGF relative to non-pre-cancerous cells having the tissue type of said pre-cancerous cells.


SEQUENCE LISTING (44 sequences)

SEQ ID NO: 1 - protein, 19 aa, Homo sapiens
  an epitope in a PCDGF K19T peptide
KKVIAPRRLPDPQILKSDT

SEQ ID NO: 2 - protein, 14 aa, Homo Sapiens
  S14R peptide
SARGTKCLRKKIPR

SEQ ID NO: 3 - protein, 19 aa, Homo sapiens
  E19V peptide
EKAPAHLSLPDPQALKRDV

SEQ ID NO: 4 - protein, 15 aa, Homo sapiens
  linker sequences inserted between identical VH and VL domains
GGGGSGGGGSGGGGS

SEQ ID NO: 5 - protein, 15 aa, Homo sapiens
  linker sequences inserted between identical VH and VL domains
ESGRSGGGGSGGGGS

SEQ ID NO: 6 - protein, 14 aa, Homo sapiens
  linker sequences inserted between identical VH and VL domains
EGKSSGSGSESKST

SEQ ID NO: 7 - protein, 15 aa, Homo sapiens
  linker sequences inserted between identical VH and VL domains
EGKSSGSGSESKSTQ

SEQ ID NO: 8 - protein, 14 aa, Homo sapiens
  linker sequences inserted between identical VH and VL domains
EGKSSGSGSESKVD

SEQ ID NO: 9 - protein, 14 aa, Homo sapiens
  linker sequences inserted between identical VH and VL domains
GSTSGSGKSSEGKG

SEQ ID NO: 10 - protein, 18 aa, Homo sapiens
  linker sequences inserted between identical VH and VL domains
KESGSVSSEQLAQFRSLD

SEQ ID NO: 11 - protein, 16 aa, Homo sapiens
  linker sequences inserted between identical VH and VL domains
ESGSVSSEELAFRSLD

SEQ ID NO: 12 - protein, 4 aa, Homo sapiens
  localization signal used to direct intrabody to endoplasmic reticulum
KDEL

SEQ ID NO: 13 - protein, 4 aa, Homo sapiens
  localization signal used to direct intrabody to endoplasmic reticulum
DDEL

SEQ ID NO: 14 - protein, 4 aa, Homo sapiens
  localization signal used to direct intrabody to endoplasmic reticulum
DEEL

SEQ ID NO: 15 - protein, 4 aa, Homo sapiens
  localization signal used to direct intrabody to endoplasmic reticulum
QEDL

SEQ ID NO: 16 - protein, 4 aa, Homo sapiens
  localization signal used to direct intrabody to endoplasmic reticulum
RDEL

SEQ ID NO: 17 - protein, 7 aa, Homo sapiens
  localization signal used to direct intrabody to nucleus
PKKKRKV

SEQ ID NO: 18 - protein, 7 aa, Homo sapiens
  localization signal used to direct intrabody to nucleus
PQKKIKS

SEQ ID NO: 19 - protein, 5 aa, Homo sapiens
  localization signal used to direct intrabody to nucleus
QPKKP

SEQ ID NO: 20 - protein, 4 aa, Homo sapiens
  localization signal used to direct intrabody to nucleus
RKKR

SEQ ID NO: 21 - protein, 5 aa, Homo sapiens
  localization signal used to direct intrabody to nucleus
KKKRK

SEQ ID NO: 22 - protein, 12 aa, Homo sapiens
  localization signal used to direct intrabody to nucleolar region
RKKRRQRRRAHQ

SEQ ID NO: 23 - protein, 16 aa, Homo sapiens
  localization signal used to direct intrabody to nucleolar region
RQARRNRRRRWRERQR

SEQ ID NO: 24 - protein, 19 aa, Homo sapiens
  localization signal used to direct intrabody to nucleolar region
MPLTRRRPAASQALAPPTP

SEQ ID NO: 25 - protein, 15 aa, Homo sapiens
  localization signal used to direct intrabody to endosomal compartment
MDDQRDLISNNEQLP

SEQ ID NO: 26 - protein, 32 aa, Homo sapiens
  localization signal used to direct intrabody to mitochondrial matrix
  VARIANT 7, 8, 32: Xaa = Any Amino Acid
MLFNLRXXLNNAAFRHGHNFMVRNFRCGQPLX

SEQ ID NO: 27 - protein, 3 aa, Homo sapiens
  localization signal used to direct intrabody to peroxisome
AKL

SEQ ID NO: 28 - protein, 6 aa, Homo sapiens
  localization signal used to direct intrabody to trans golgi network
SDYQRL

SEQ ID NO: 29 - protein, 8 aa, Homo sapiens
  localization signal used to direct intrabody to plasma membrane
GCVCSSNP

SEQ ID NO: 30 - protein, 8 aa, Homo sapiens
  localization signal used to direct intrabody to plasma membrane
GQTVTTPL

SEQ ID NO: 31 - protein, 8 aa, Homo sapiens
  localization signal used to direct intrabody to plasma membrane
GQELSQHE

SEQ ID NO: 32 - protein, 8 aa, Homo sapiens
  localization signal used to direct intrabody to plasma membrane
GNSPSYNP

SEQ ID NO: 33 - protein, 8 aa, Homo sapiens
  localization signal used to direct intrabody to plasma membrane
GVSGSKGQ

SEQ ID NO: 34 - protein, 8 aa, Homo sapiens
  localization signal used to direct intrabody to plasma membrane
GQTITTPL

SEQ ID NO: 35 - protein, 8 aa, Homo sapiens
  localization signal used to direct intrabody to plasma membrane
GQTLTTPL

SEQ ID NO: 36 - protein, 8 aa, Homo sapiens
  localization signal used to direct intrabody to plasma membrane
GQIFSRSA

SEQ ID NO: 37 - protein, 8 aa, Homo sapiens
  localization signal used to direct intrabody to plasma membrane
GQIHGLSP

SEQ ID NO: 38 - protein, 8 aa, Homo sapiens
  localization signal used to direct intrabody to plasma membrane
GARASVLS

SEQ ID NO: 39 - protein, 8 aa, Homo sapiens
  localization signal used to direct intrabody to plasma membrane
GCTLSAEE

SEQ ID NO: 40 - protein, 16 aa, Homo sapiens
  membrane permeable sequence
AAVALLPAVLLALLAP

SEQ ID NO: 41 - protein, 12 aa, Homo sapiens
  membrane permeable sequence
AAVLLPVLLAAP

SEQ ID NO: 42 - protein, 15 aa, Homo sapiens
  membrane permeable sequence
VTVLALGALAGVGVG

SEQ ID NO: 43 - DNA, 21 bp, Artificial Sequence
  antisense molecule directed to PCDGF
gggtccacatggtctgcctgc

SEQ ID NO: 44 - DNA, 24 bp, Artificial Sequence
  antisense molecule directed to PCDGF
gccaccagccctgctgttaaggcc